(19) United States
(12) Patent Application Publication
(10) Pub. No.: US 2017/0232054 A1
(43) Pub. Date: Aug. 17, 2017

(54) Method for economically producing stable and bio-available glutathione

(71) Applicants: Wilson M. Shepard, San Antonio, TX (US); Brad Blum, Sugar Land, TX (US)

(72) Inventors: Wilson M. Shepard, San Antonio, TX (US); Brad Blum, Sugar Land, TX (US)

(21) Appl. No.: 15/400,505

(22) Filed: Jan. 6, 2017

Related U.S. Application Data
(60) Provisional application No. 62/275,614, filed on Jan. 6, 2016.

(51) Int. Cl. A61K 38/06; A61K 9/00; A61K 9/70; A61K 9/14

(52) U.S. Cl. CPC A61K 38/063; A61K 9/148; A61K 9/145; A61K 9/0014; A61K 9/7023

(57) ABSTRACT

A method of economically producing stable and bio-available glutathione comprising immersing glutathione particles in an edible oil preferably selected from at least one of almond, coconut, grapeseed and olive; reducing the average size of glutathione particles to not more than 50 microns and preferably 1 or less; and adding a de-agglomeration agent comprising at least one selected from surfactants or chelating agents. A method of administering bio-available glutathione, prepared according to the foregoing, to a patient in a desired dose by a method selected from oral, pulmonary, rectal, sublingual, transdermal, and vaginal.

### CROSS-REFERENCE TO RELATED APPLICATIONS

This application claims priority of provisional Patent Application No. 62/275,614 filed Jan. 6, 2016.

### FIELD OF THE INVENTION

The present invention generally relates to processing methods for glutathione. In particular, the invention relates to methods for increasing the stability and bio-availability of glutathione while containing production costs.

### BACKGROUND

Glutathione is an antioxidant that has been shown to prevent damage to cells otherwise caused by reactive oxygen species and by heavy metals. Glutathione is present in both plants and animals, and it is crucial to biological processes in both. Low glutathione levels in humans are often indicative of adverse health conditions as diverse as cancer, burns and athletic overtraining. Glutathione can prevent oxidative damage to the skin, and it also can lighten skin pigmentation. Therefore, glutathione is often a desirable component to be included in lotions for topical human application.

More broadly, it is desirable to treat many conditions in humans with glutathione. Problems with such treatments in the past included the instability of glutathione—particularly its tendency to oxidize. The tendency to oxidize or otherwise degrade means that products containing glutathione have poor shelf life.

Glutathione has also not been effective as an oral human supplement because of its poor bioavailability. Poor bioavailability is due to both destruction of glutathione in the alimentary canal (digestive tract) and the absence of a carrier of glutathione through cellular membranes.

The prior art has envisioned resizing glutathione particles and even stabilizing it by combination with other compounds to increase its bioavailability. For example, Patel (US Pub. No. 2012/042997; WO 2012174555A) discussed reducing glutathione particle size using a diamond chamber, but that was not the real focus of the invention. Patel chemically bound glutathione to another compound, and its size in that chemically-bound state was reported to be 7.5-8.15 angstroms in diameter. An angstrom is very small: there are 10,000 micrometers (commonly referred to as microns) in an angstrom. As can be seen from the discussion of the invention below, the particle size of glutathione in the present invention is orders of magnitude greater than for Patel.

Patel also discussed that cyclodextrin-encapsulation and reduction of glutathione particles to the cited size range allowed the glutathione to be absorbed through the skin or other membranes, though it teaches away from oral administration. Patel teaches that treatment of glutathione with cyclodextrins, comprising sugar molecules bound together in a ring having an inner portion able to host hydrophobic molecules (such as glutathione). The cyclodextrin is purported to be absorbed through the skin at which point the cyclodextrins are broken down freeing the glutathione to be absorbed into the bloodstream. Oral administration of Patel's formulation is not effective because the sugar ring is dissolved, and the exposed glutathione is simply digested.

Patel's process is quite complicated. Due to the complication of the process required to create a sufficient yield of cyclodextrin-encapsulated glutathione, resulting products are relatively expensive.

Wang et al. (U.S. Pat. No. 8,569,239) taught the use of grinding beads to create a sticky paste, but not to resize the individual particles of components contained therein. Further, the mixture in Wang did not include glutathione per se, but rather glutathione in a complex with other compounds such a α-tocopherol succinate PEG 1500 (TPGS) as a small percentage (less than 10%) of the total mixture. Wang also taught use of lecithin as a carrier, but only in a cream containing primarily lecithin. Wang taught the need for a “carrier” compound present in much greater quantities than the glutathione with which it was to be bound.

In unrelated art, it was discovered that particles in the range of 0.1-1.0 microns can be absorbed into cells of the lungs. Geiser et al, “Ultrafine Particles Cross Cellular Membranes by Nonphagocytic Mechanisms in Lungs and Cultured Cells,” ENVIRON. HEALTH PERSPECT., November 2005 (113(11)). That discovery presented the possibility that desired materials could be delivered across cellular boundaries by appropriate sizing of particles to be delivered. However, teachings in the prior art such as Patel (U.S. Pub. No. 2012/042997) have assumed that particle size must be on the angstrom level to allow glutathione to cross cellular boundaries.

There has been a need for an economical process that can provide stability for products containing glutathione and improve its bioavailability. However, prior art processes were expensive and produced complicated mixtures or chemical compounds containing glutathione along with a significant number of other compounds that are not generally desirable in treatments using glutathione. Therefore, a need exists for an economical method of delivering bioavailable glutathione in a form that has a significant shelf life.

### SUMMARY OF THE INVENTION

The invention relates to a method for economical production of bioavailable and stable glutathione in a relatively pure formulation. Glutathione is stabilized by immersion in an edible oil such as almond, coconut, grapeseed, olive, or vegetable. The size of glutathione particles is reduced by physical processes to circa an average of less than 50 microns, preferably around 1 micron, and it is combined with a de-agglomeration agent comprising a surfactant and possibly a chelating agent.

Use of lecithin is a preferred de-agglomeration agent that also serves to facilitate penetration of cellular barriers by glutathione, which is normally not carried through cell walls. The use of lecithin, which acts as a cellular-level surfactant combined with the small particle size of glutathione produced by the method makes the glutathione more bio-available.

It has been discovered that glutathione particles in the micron size range can be absorbed through cellular membranes, so the present method's ability to economically produce stable glutathione particles in that size range renders them capable of penetrating cell walls and becoming biologically beneficial within individual cells. Glutathione prepared according to the method can be delivered by multiple means—transdermal, sublingual, inhaled, oral, or rectal. Experiments have confirmed the ability of the glutathione prepared according to the method to cross a membrane simulating sublingual administration.

There have thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction and to the arrangements of the components set forth in the following description or illustrated in the drawings. The invention is capable of other embodiments and of being practiced and carried out in various ways. Also, it is to be understood that the phraseology and terminology employed herein are for generalized description, and they should not be regarded as limiting. As such, those skilled in the art will appreciate that the conception, upon which this disclosure is based, may readily be utilized as a basis for the designing of other structures, methods, and systems for carrying out the several purposes of the present invention. Additional benefits and advantages of the present invention will become apparent to those skilled in the art to which the present invention relates from the subsequent description of the preferred embodiment and the appended claims, taken in conjunction with the accompanying drawings. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Other features, aspects, and advantages will become apparent from the description, the drawings, and the claims.

### DESCRIPTION OF THE DRAWINGS

FIG. 1 shows the engagement of a de-agglomeration agent with a particle of glutathione serving both to facilitate biological uptake and to prevent agglomeration of glutathione particles.

FIG. 2 shows a batch processing vessel that may be used for reducing the average size of glutathione particles.

FIG. 3 is a table with experimental results for a batch size reduction process.

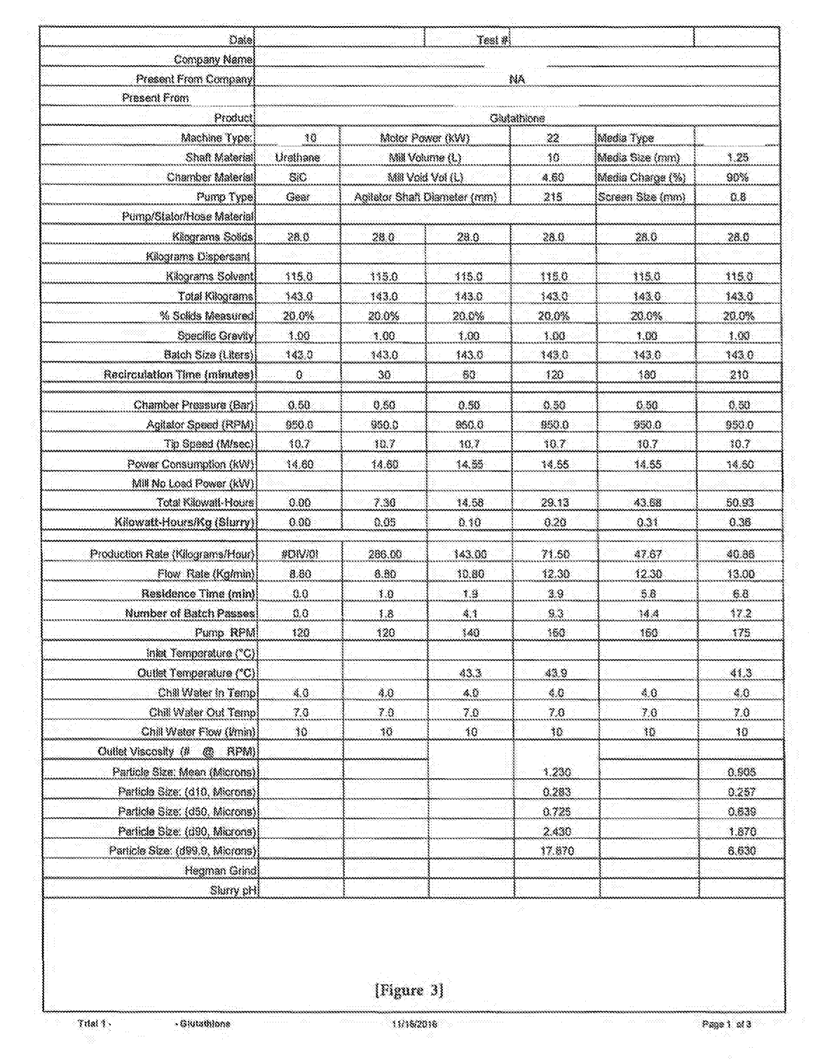

FIG. 4 is a table with experimental results for a batch size reduction process.

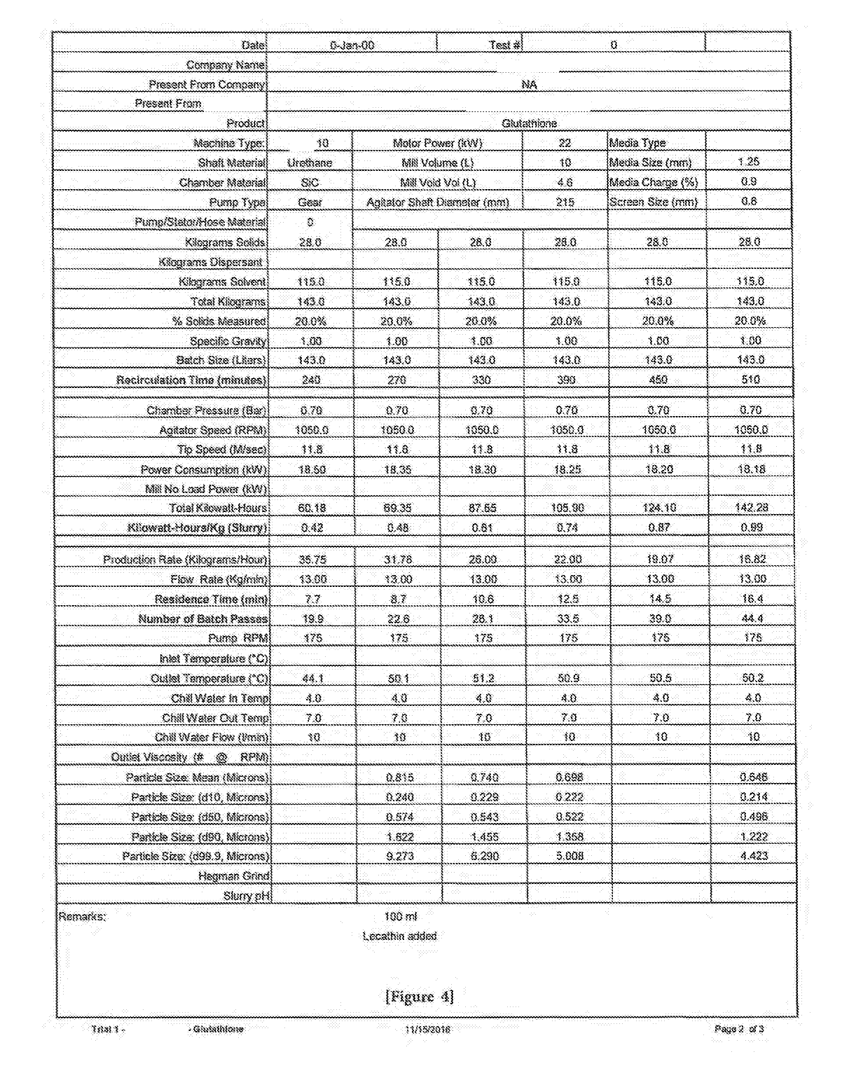

FIG. 5 is a table with experimental results for a batch size reduction process.

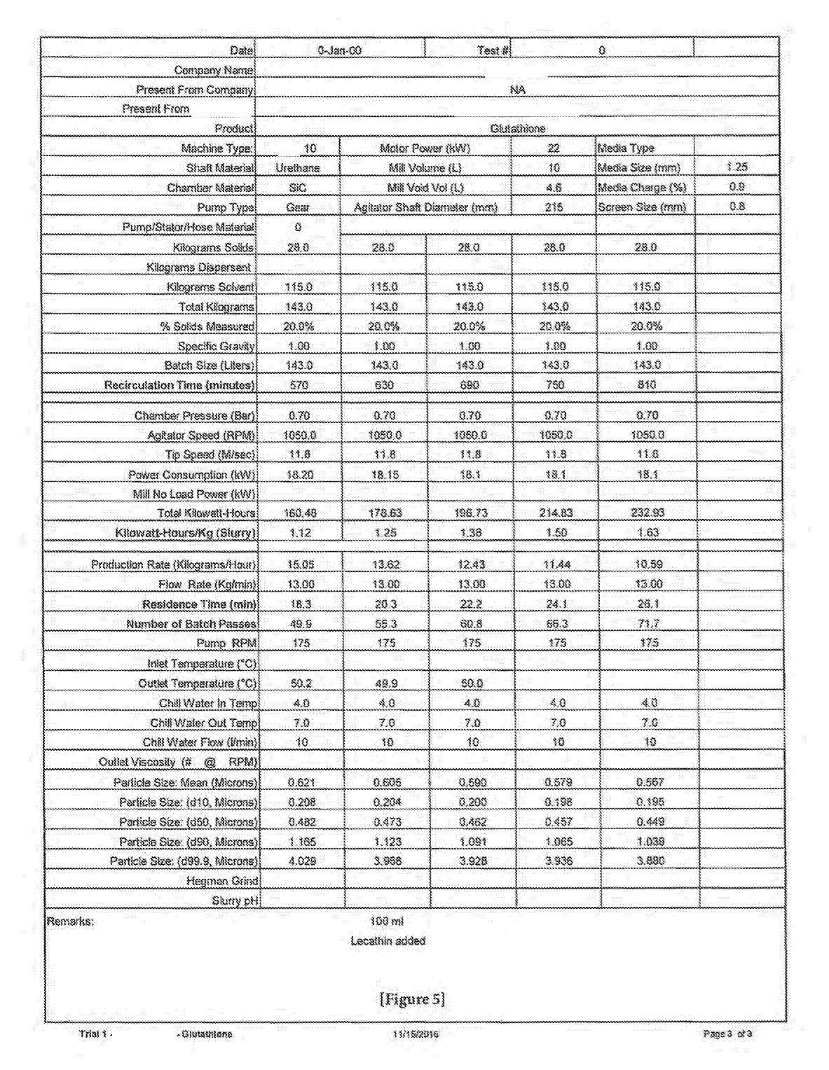

FIG. 6 is a graph showing experimental results for a batch size reduction process.

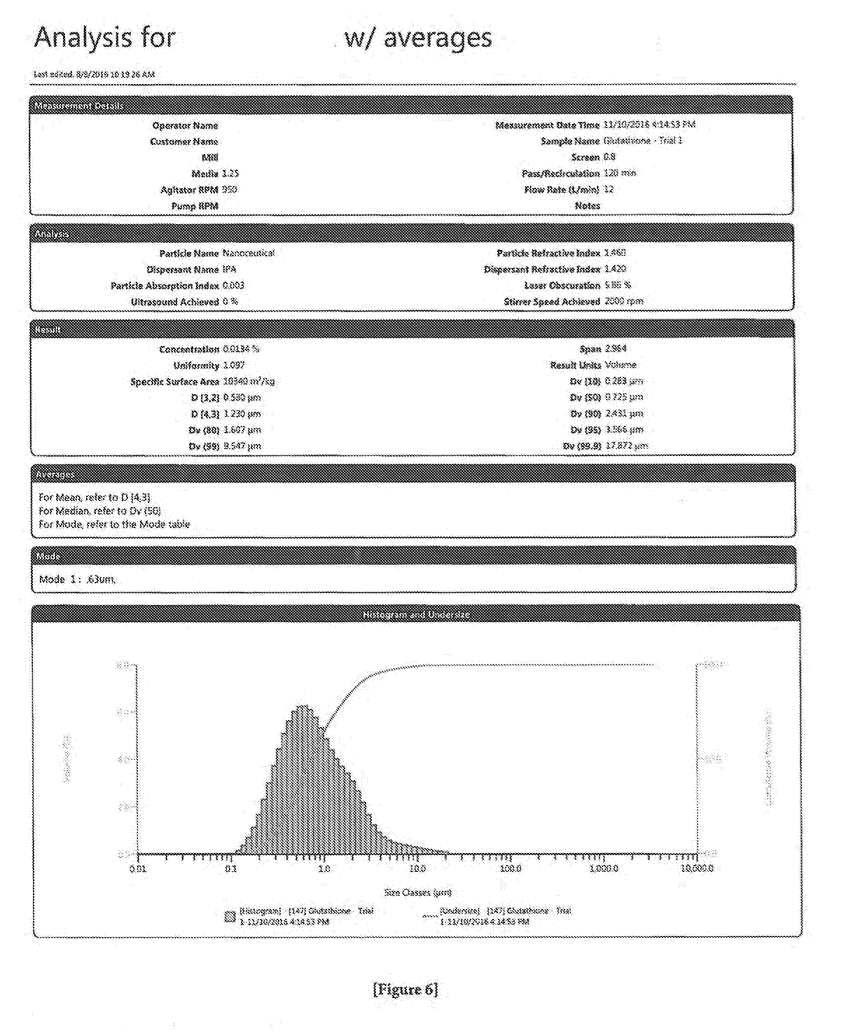

FIG. 7 is a graph showing experimental results for a batch size reduction process.

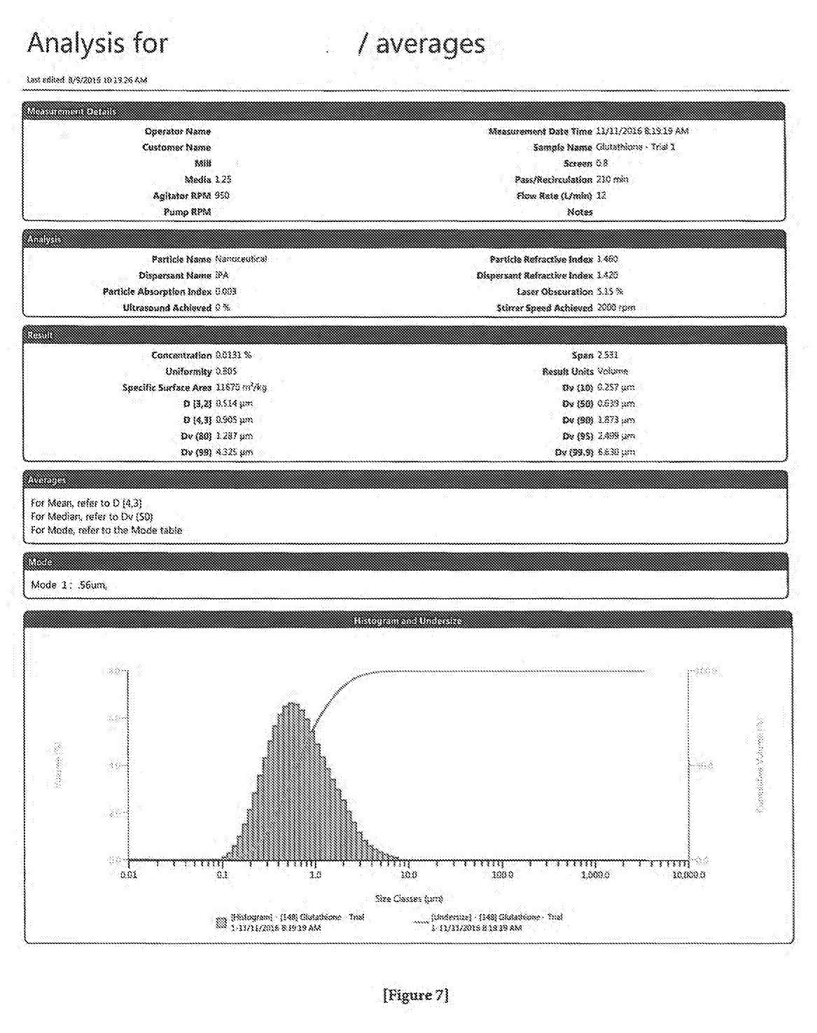

FIG. 8 is a graph showing experimental results for a batch size reduction process.

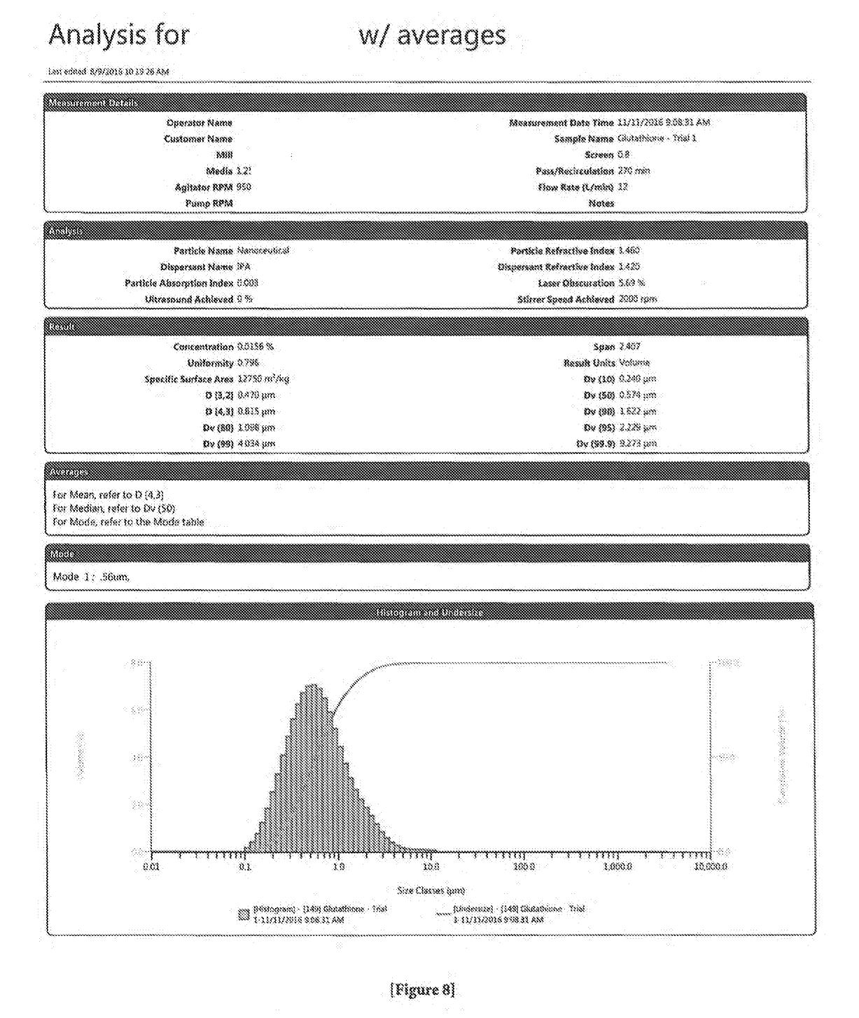

FIG. 9 is a graph showing experimental results for a batch size reduction process.

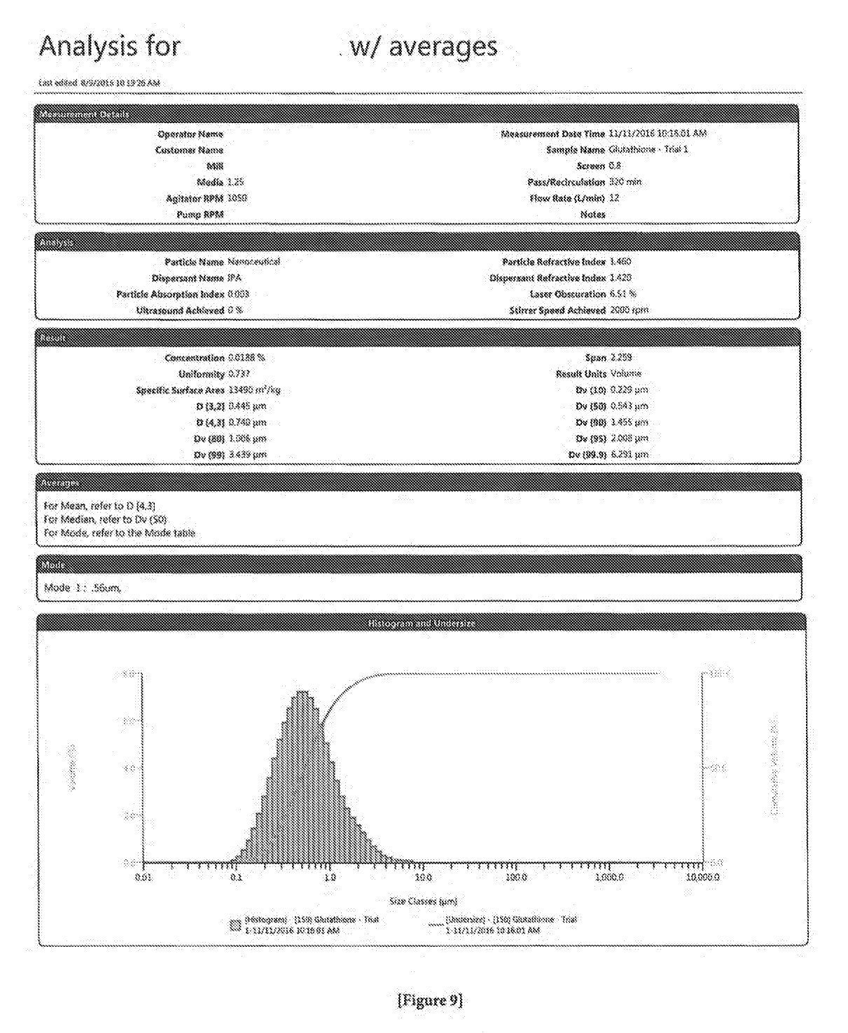

FIG. 10 is a graph showing experimental results for a batch size reduction process.

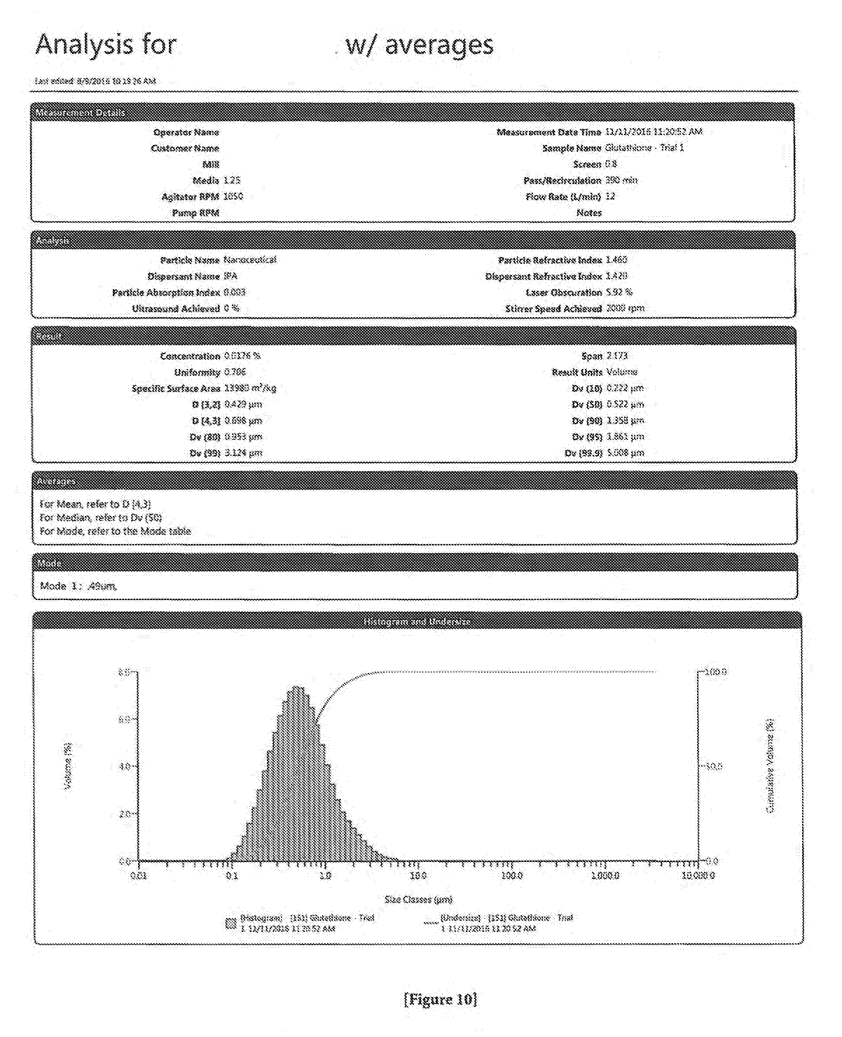

FIG. 11 is a graph showing experimental results for a batch size reduction process.

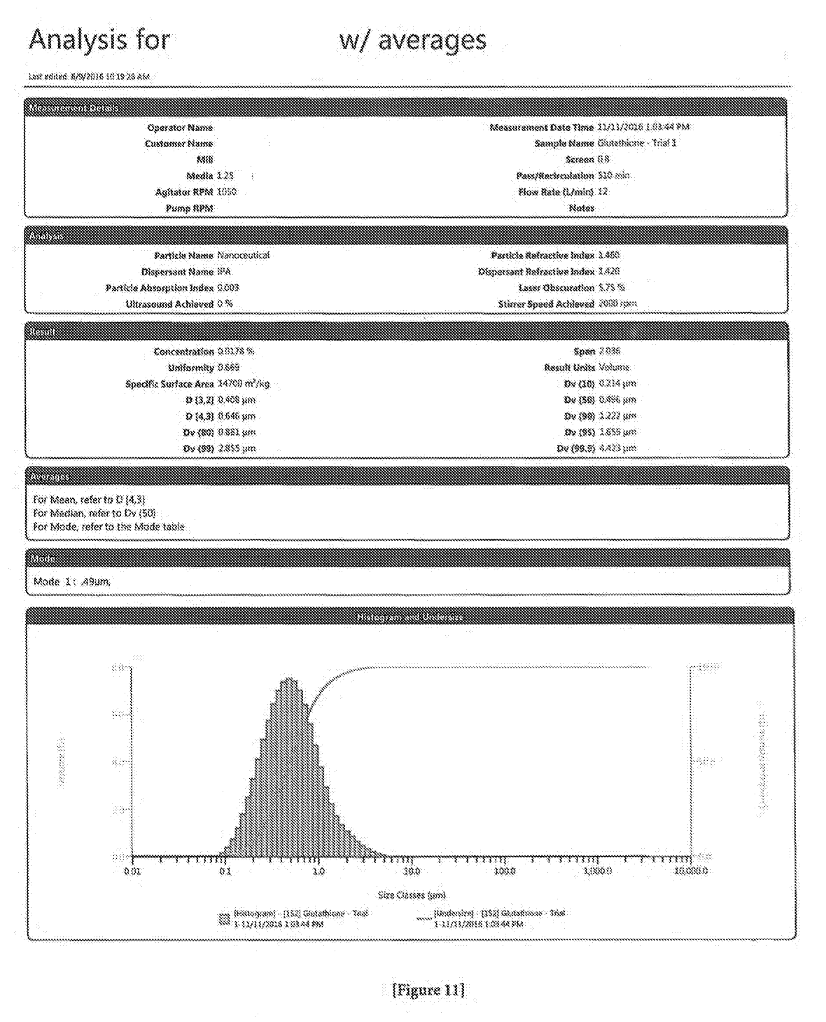

FIG. 12 is a graph showing experimental results for a batch size reduction process.

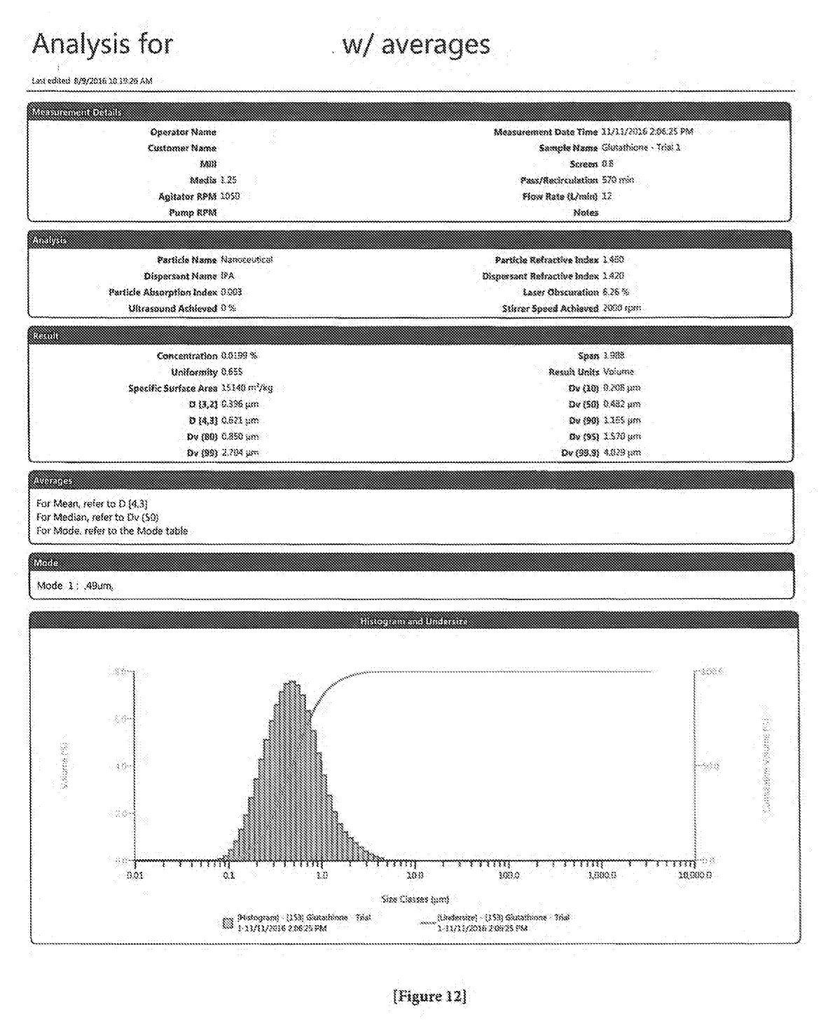

FIG. 13 is a graph showing experimental results for a batch size reduction process.

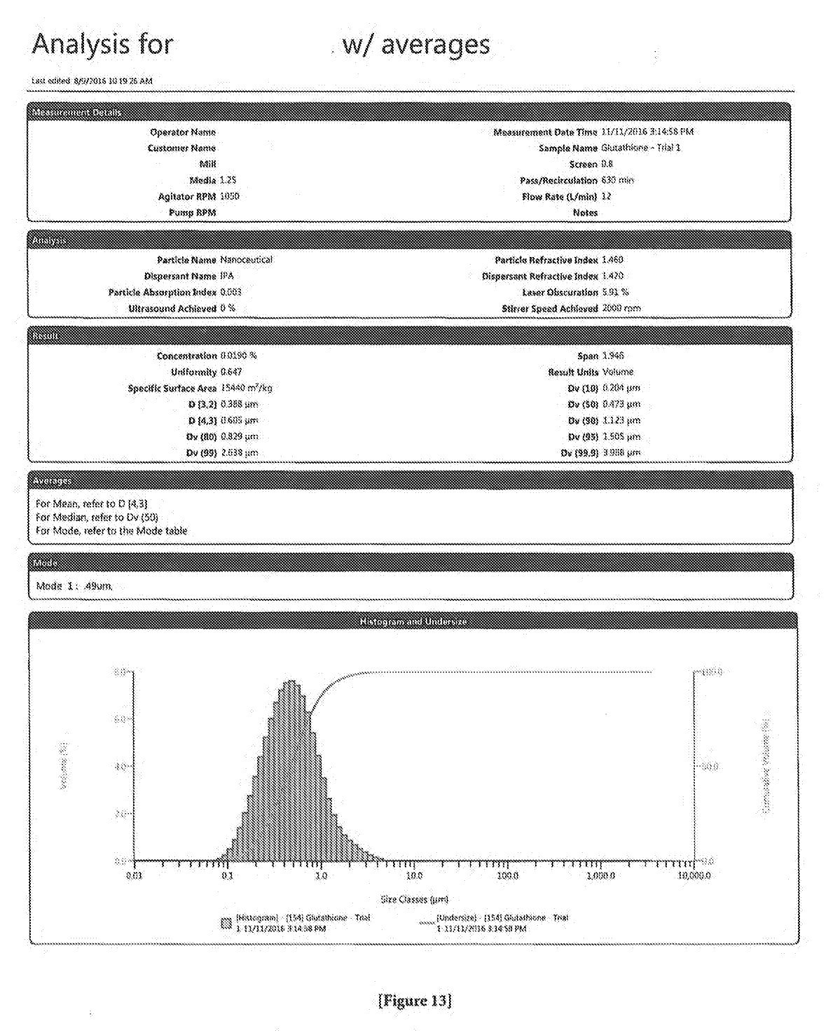

FIG. 14 is a graph showing experimental results for a batch size reduction process.

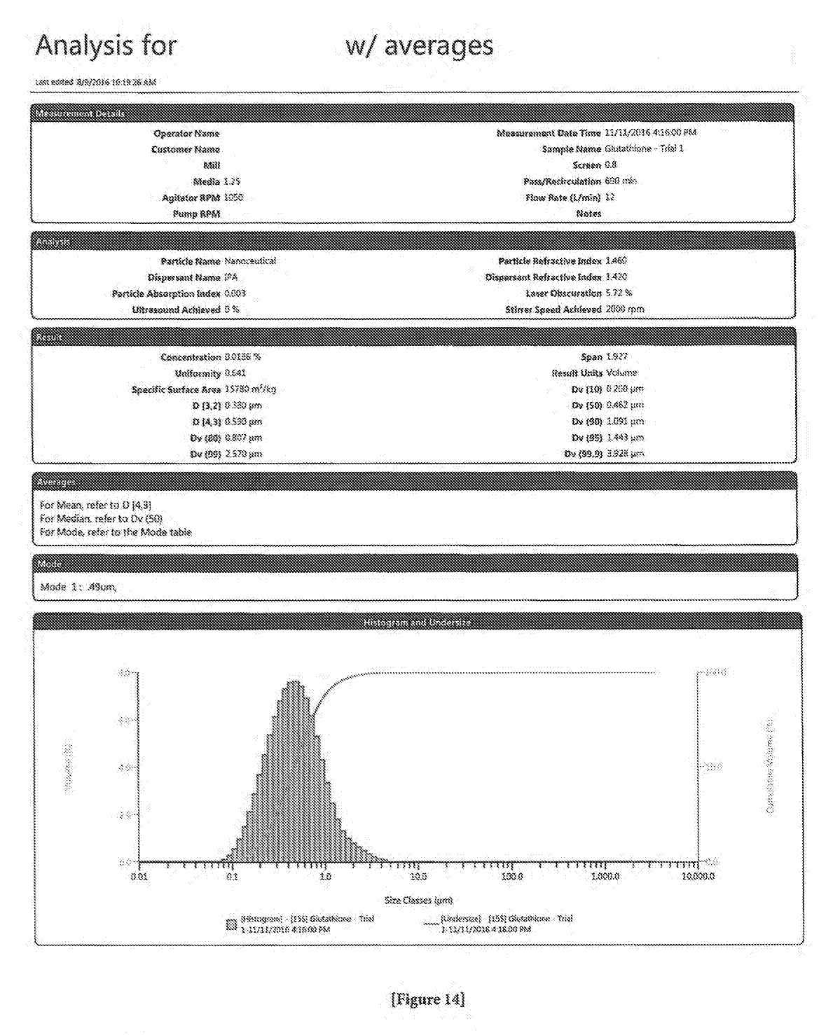

FIG. 15 is a graph showing experimental results for a batch size reduction process.

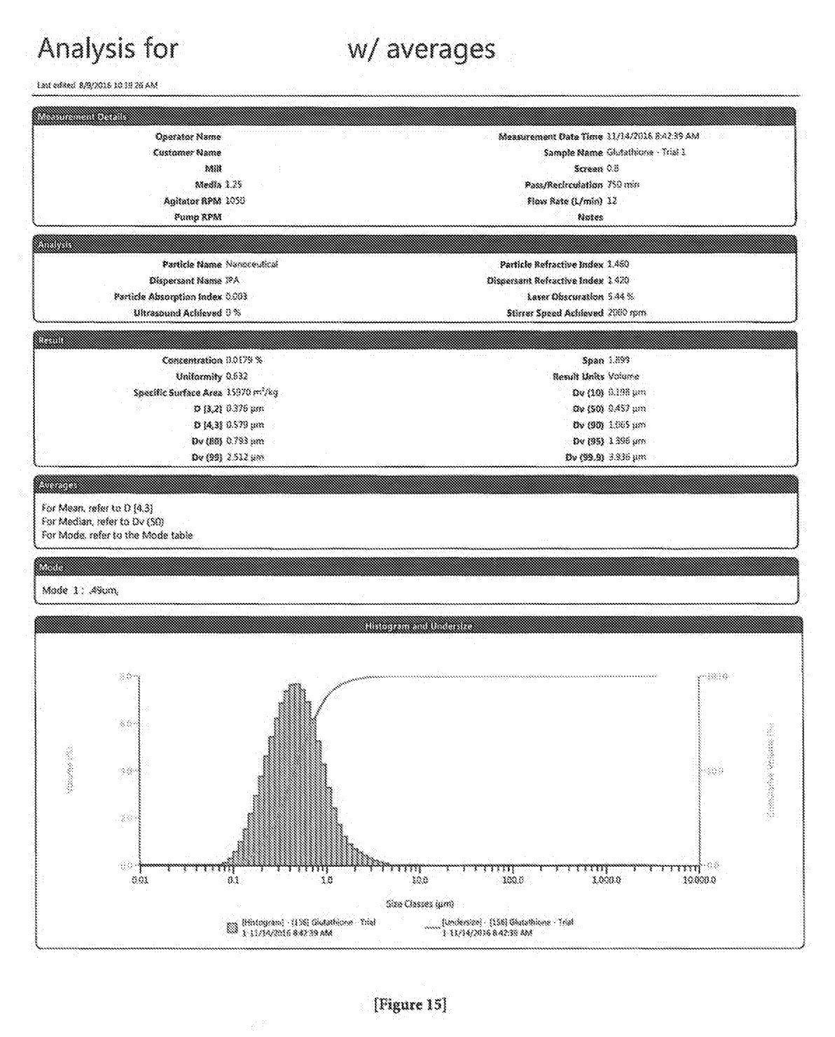

FIG. 16 is a graph showing experimental results for a batch size reduction process.

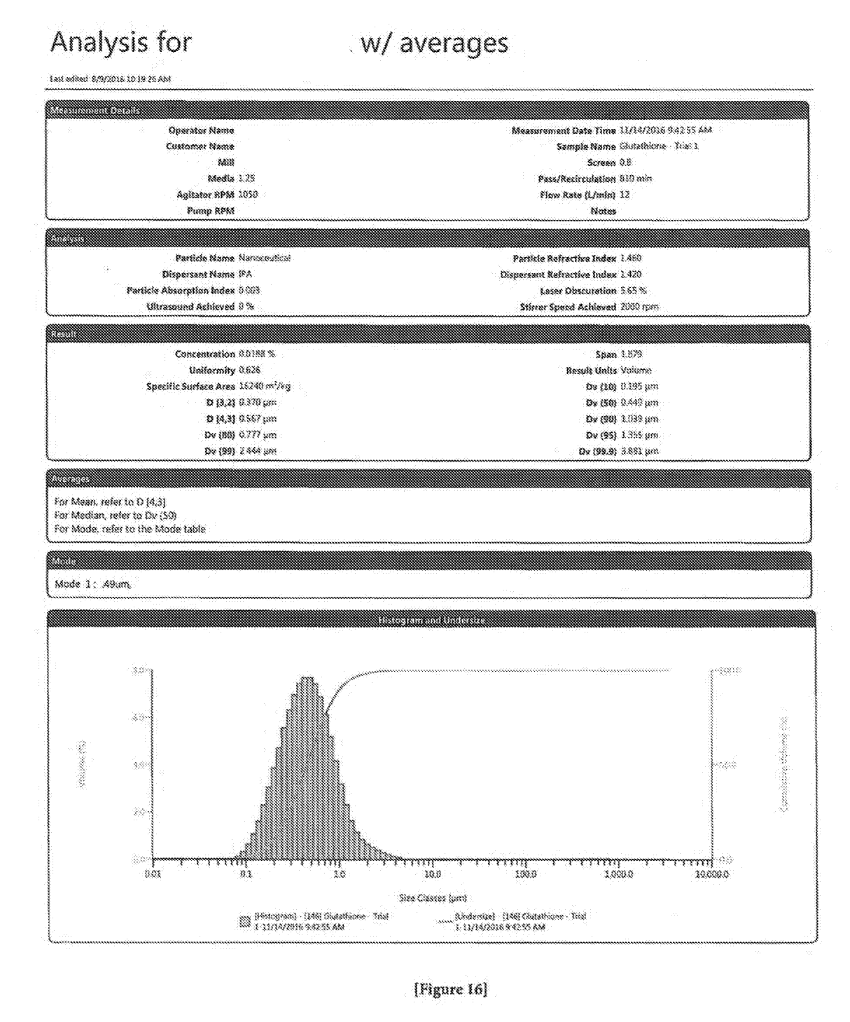

FIG. 17 is a table with experimental results for a batch size reduction process.

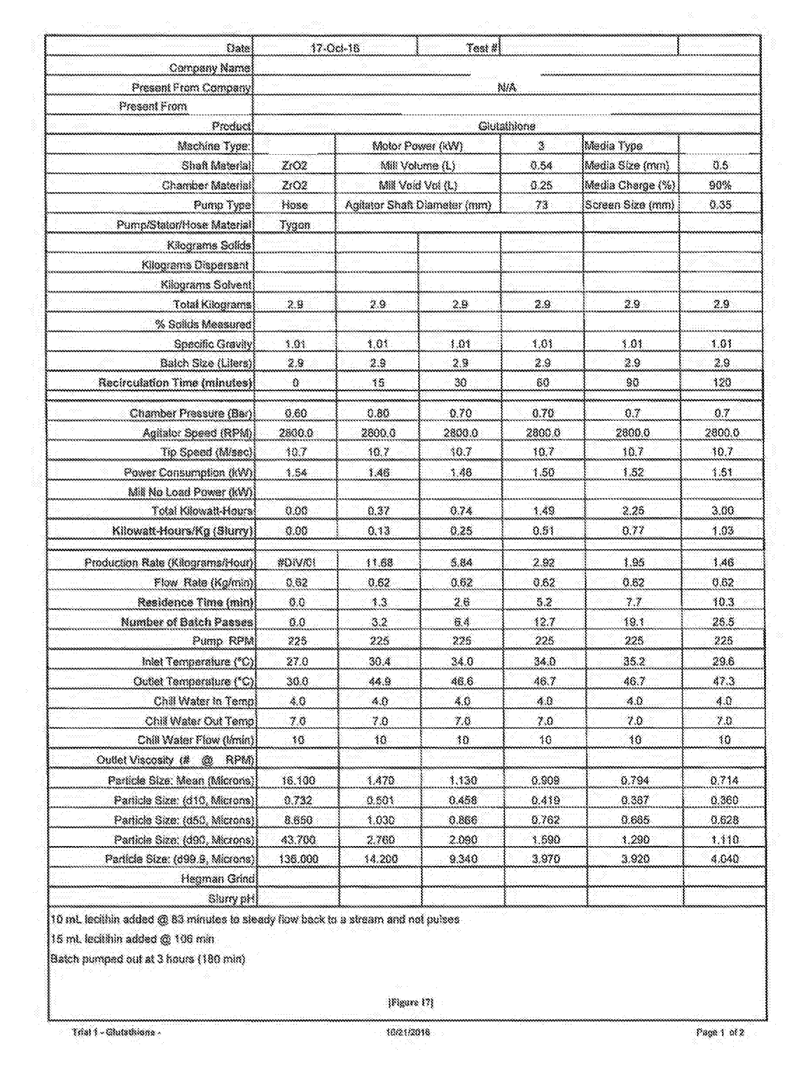

FIG. 18 is a table with experimental results for a batch size reduction process.

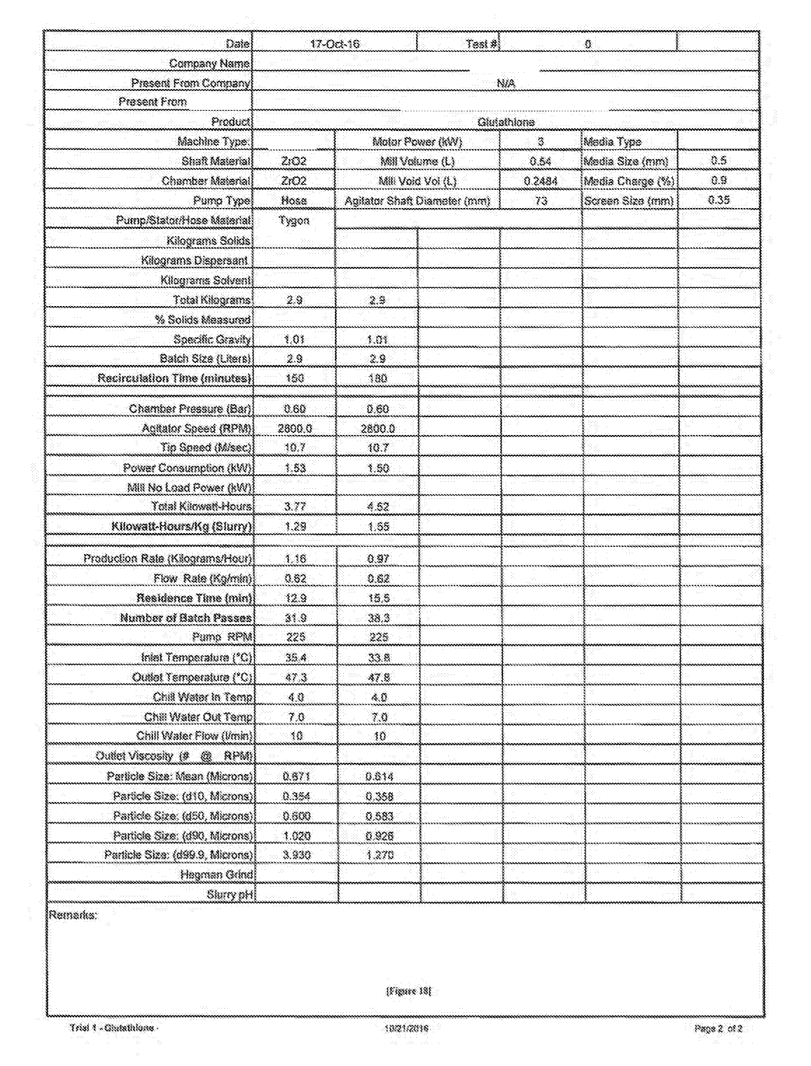

FIG. 19 is a graph showing experimental results for a batch size reduction process.

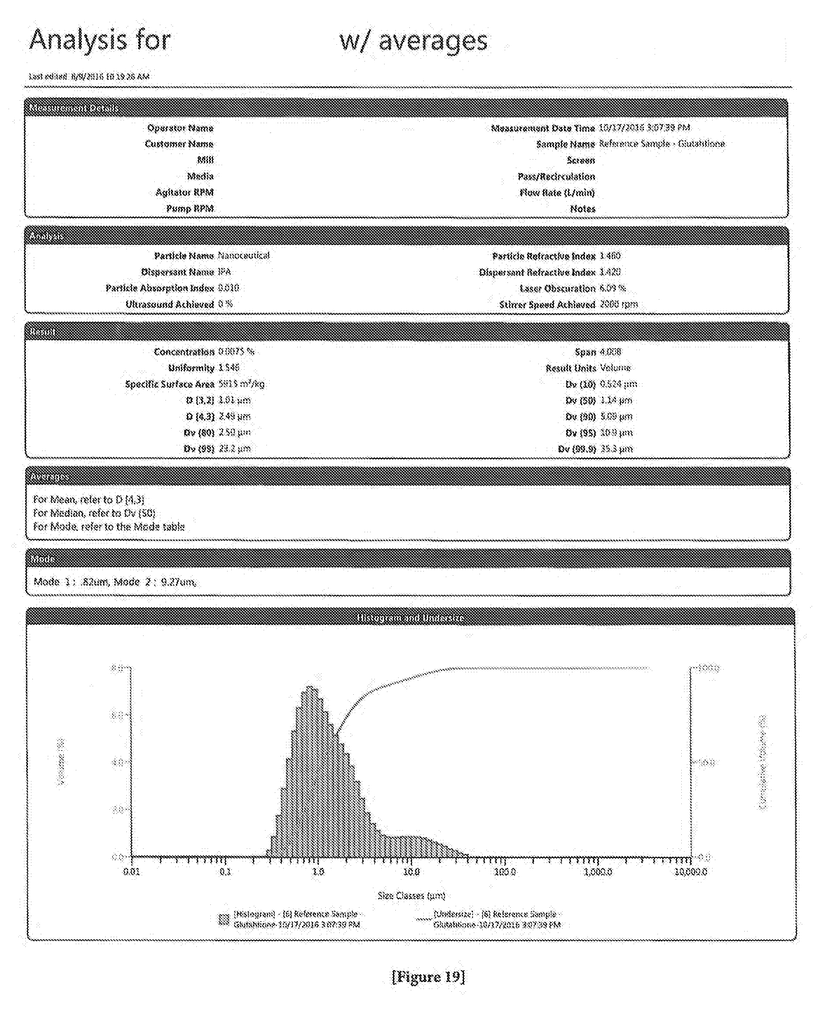

FIG. 20 is a graph showing experimental results for a batch size reduction process.

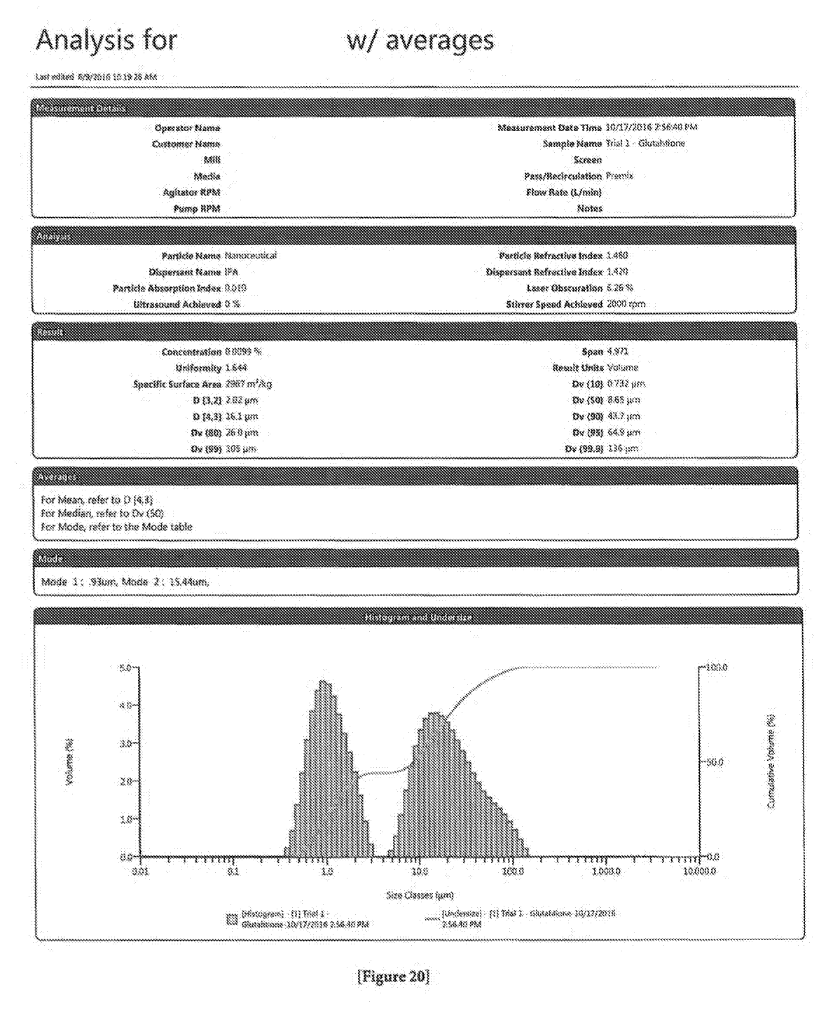

FIG. 21 is a graph showing experimental results for a batch size reduction process.

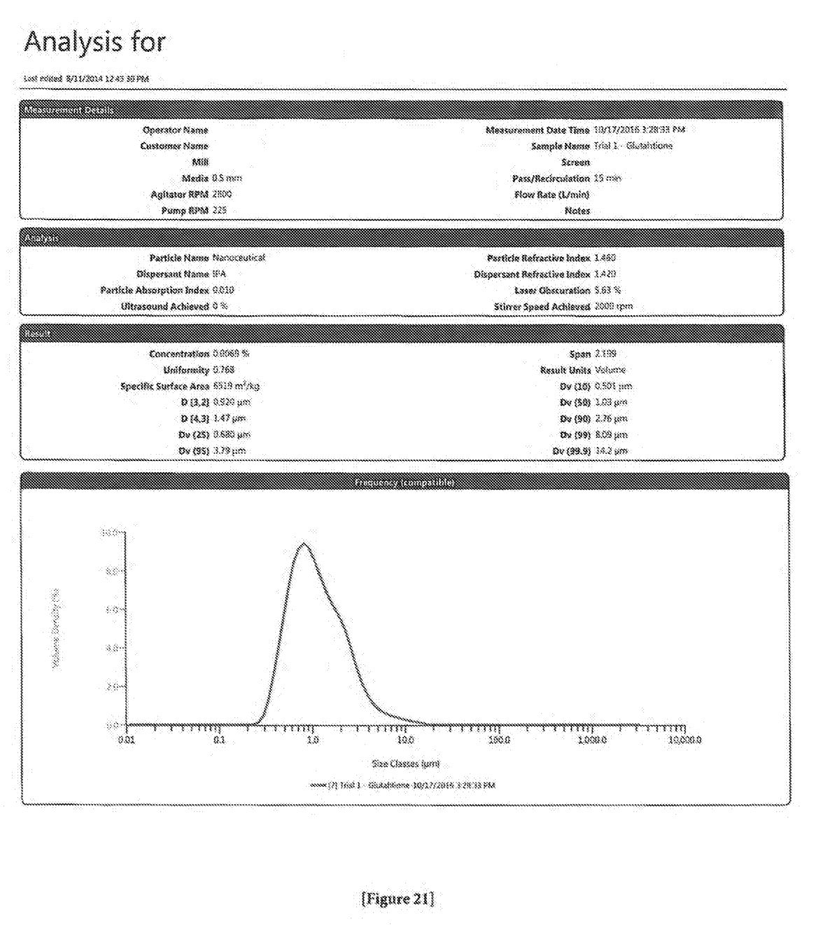

FIG. 22 is a graph showing experimental results for a batch size reduction process.

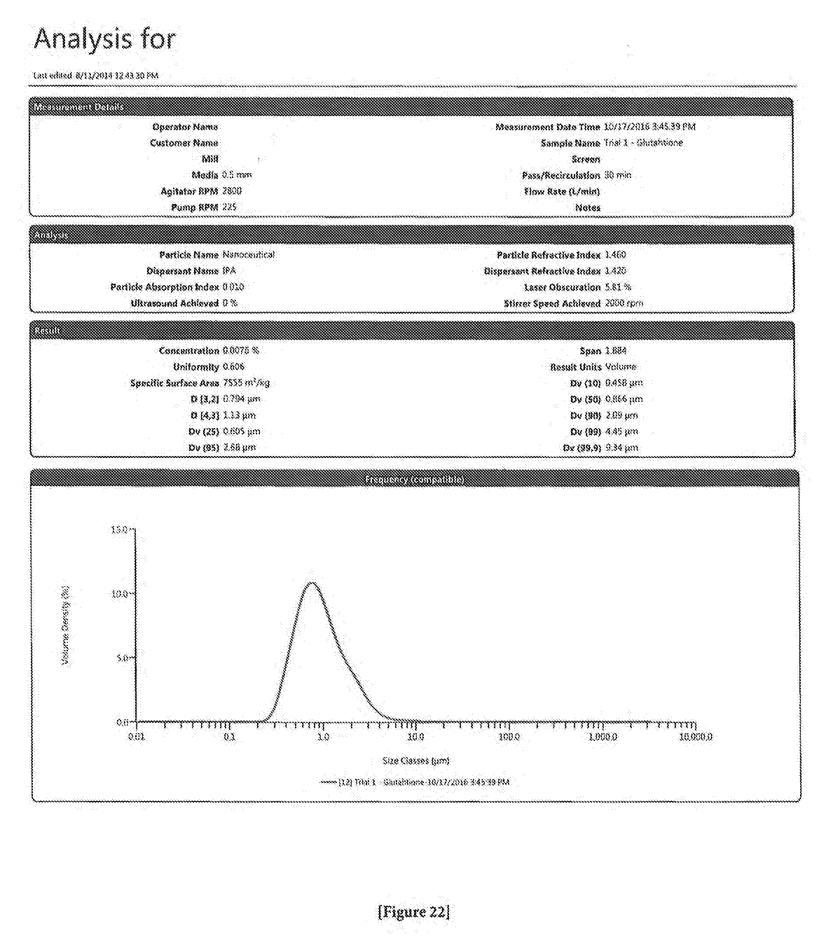

FIG. 23 is a graph showing experimental results for a batch size reduction process.

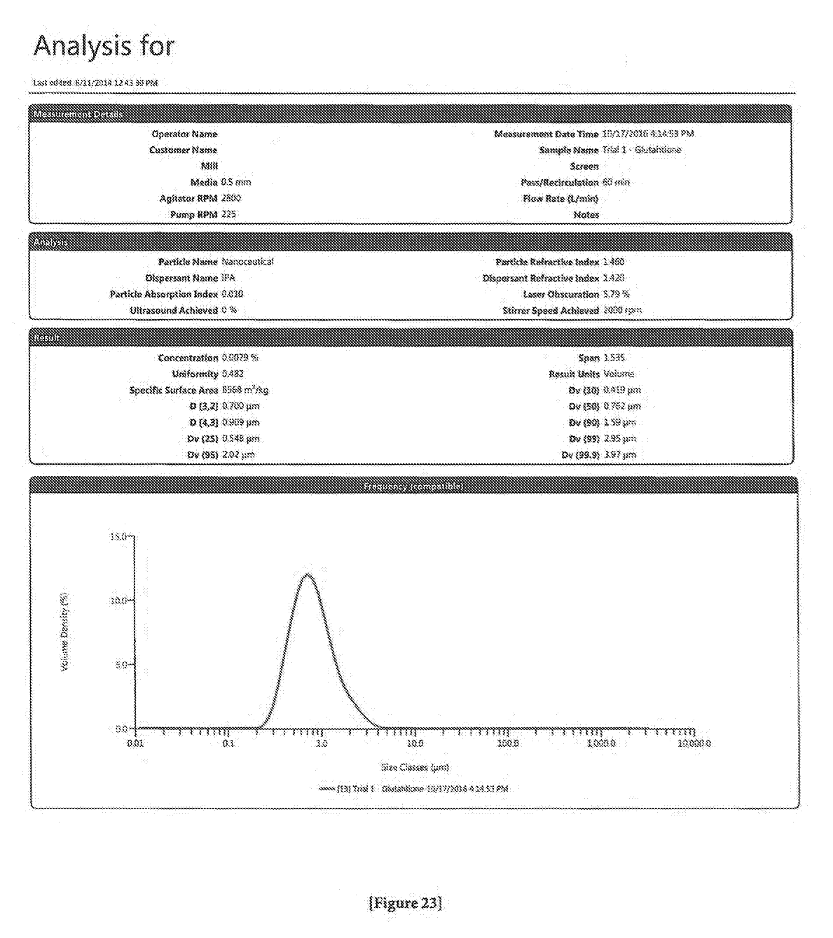

FIG. 24 is a graph showing experimental results for a batch size reduction process.

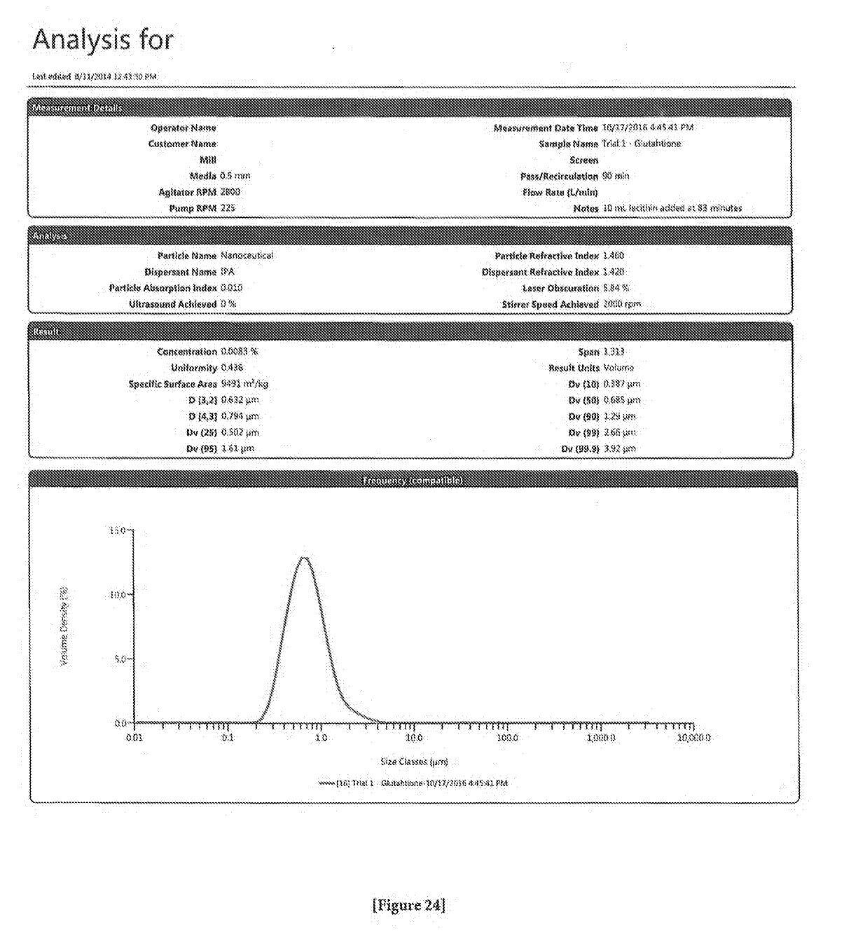

FIG. 25 is a graph showing experimental results for a batch size reduction process.

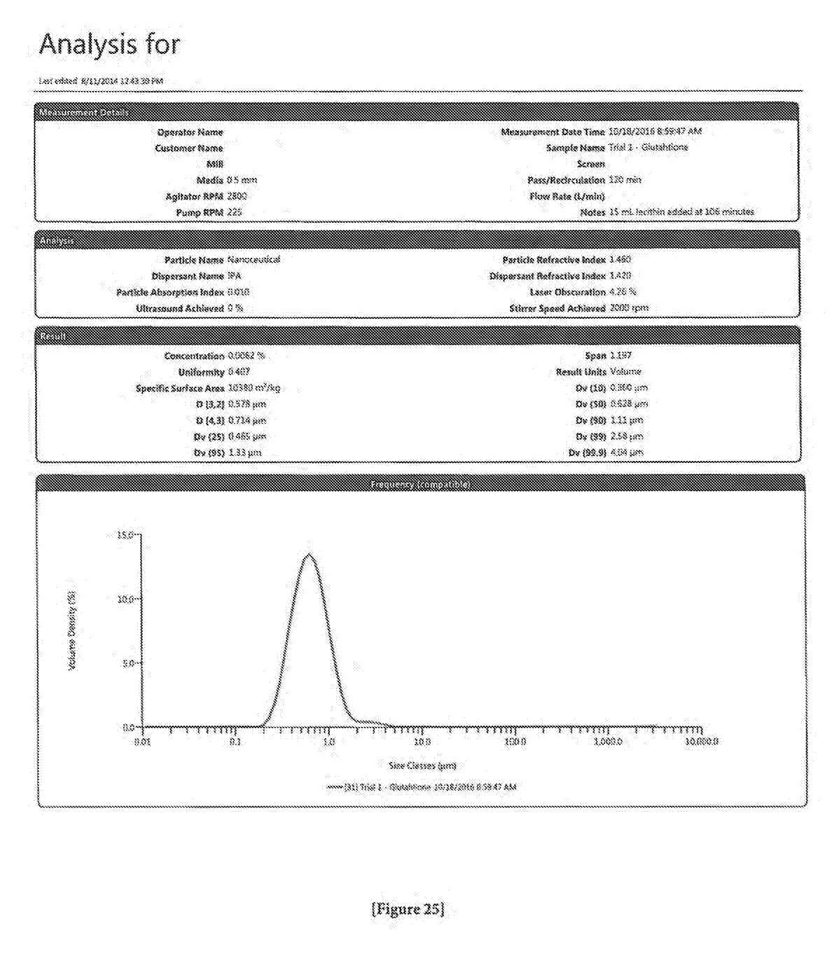

FIG. 26 is a graph showing experimental results for a batch size reduction process.

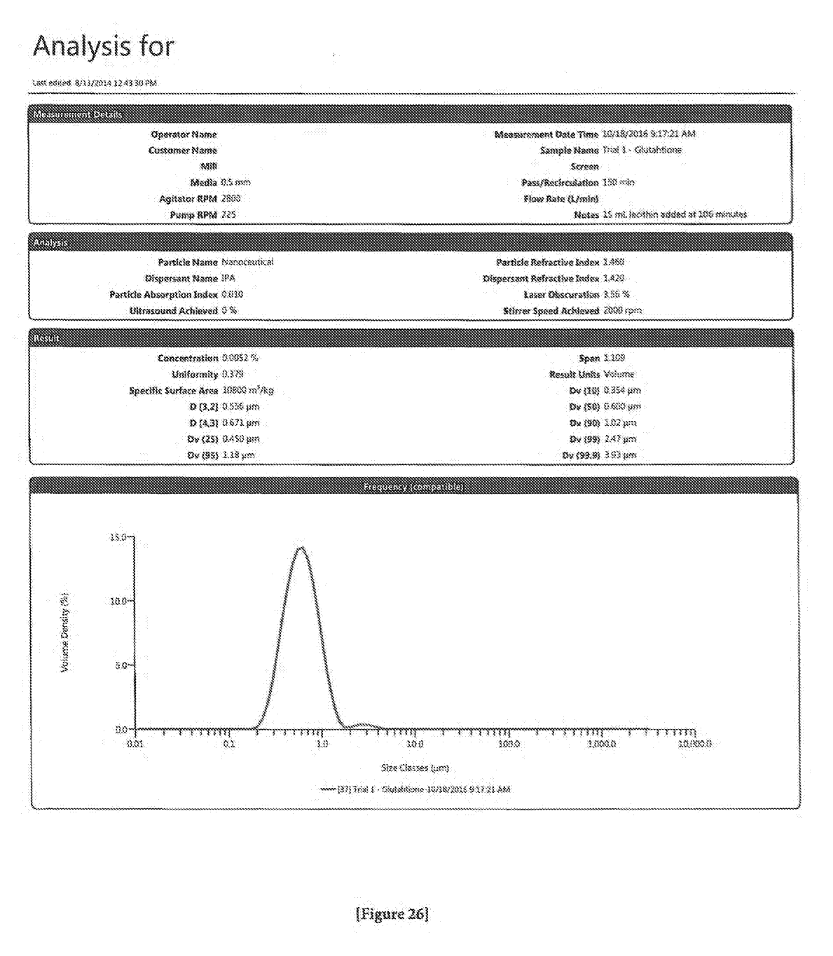

FIG. 27 is a graph showing experimental results for a batch size reduction process.

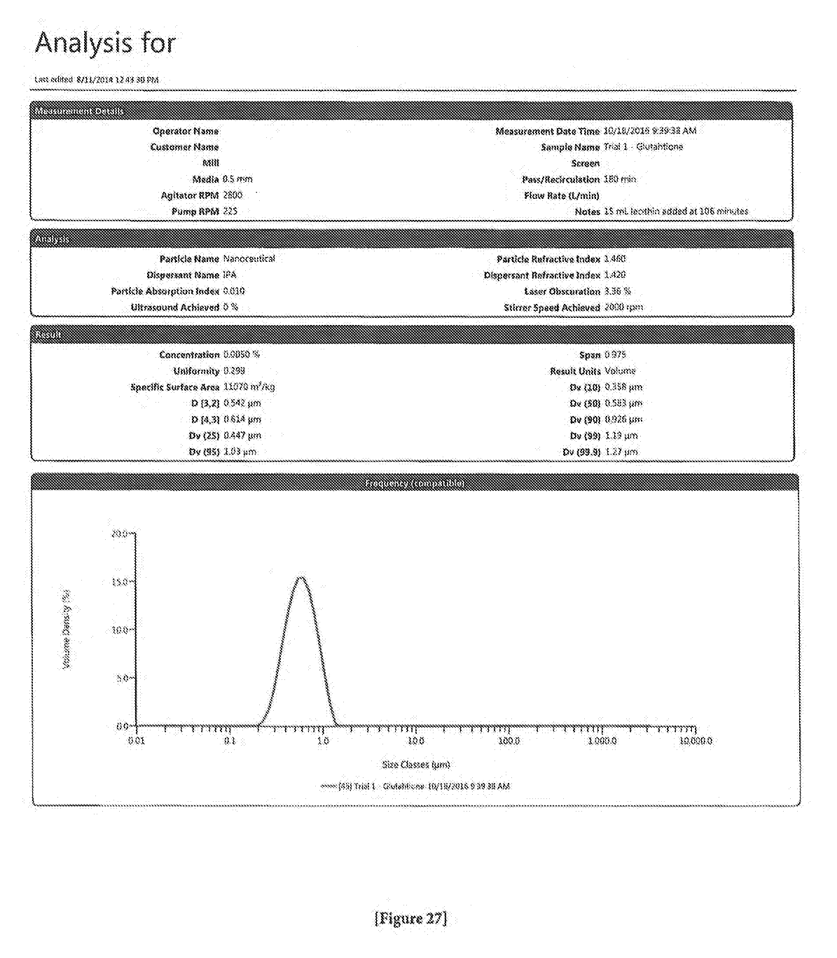

FIG. 28 is a table with experimental results for a batch size reduction process.

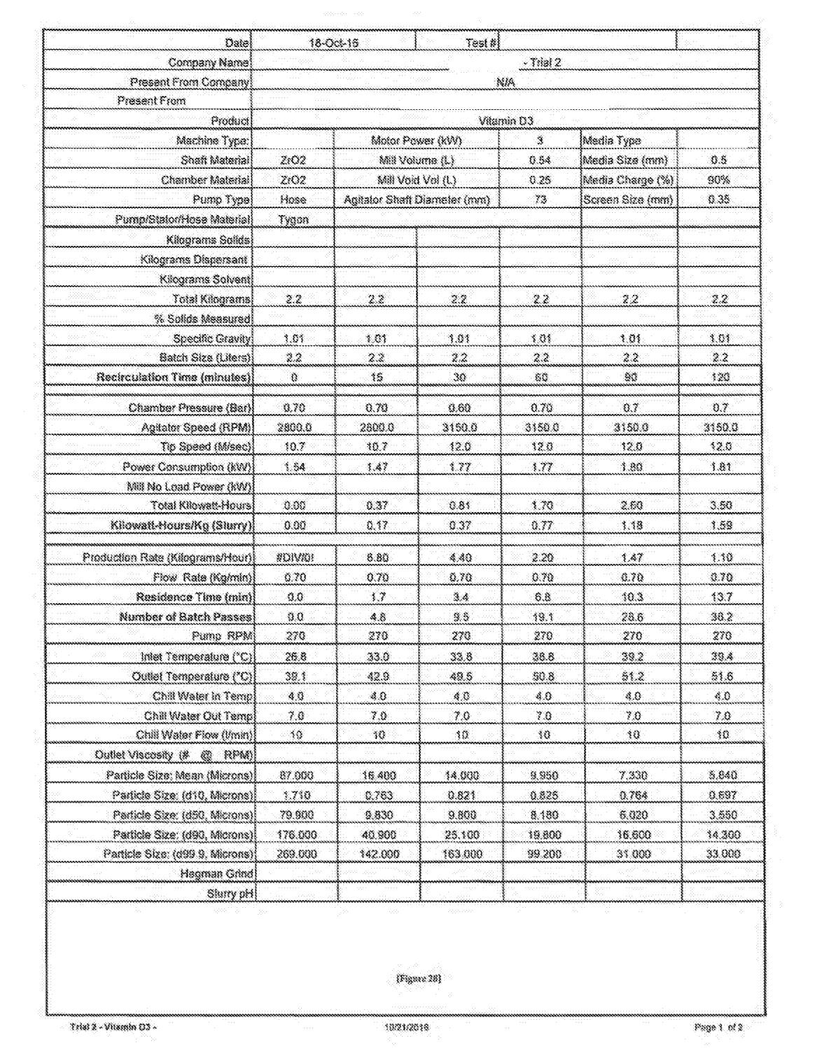

FIG. 29 is a table with experimental results for a batch size reduction process.

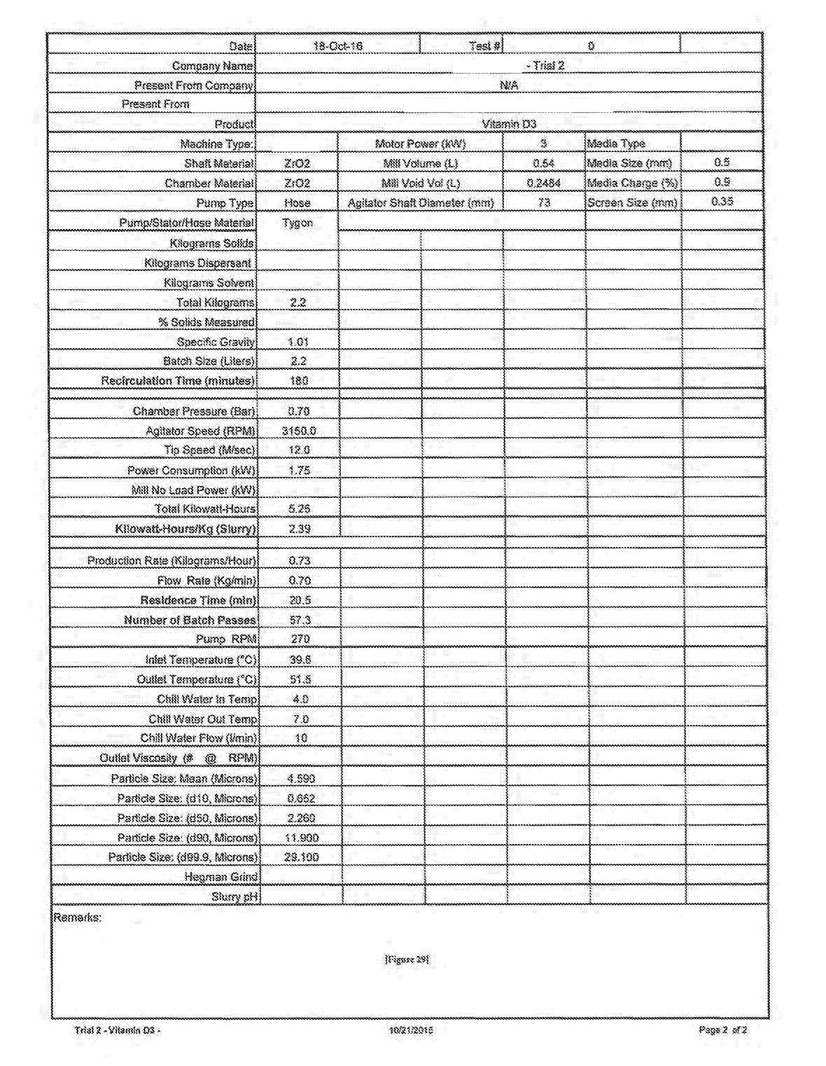

FIG. 30 is a graph showing experimental results for a batch size reduction process.

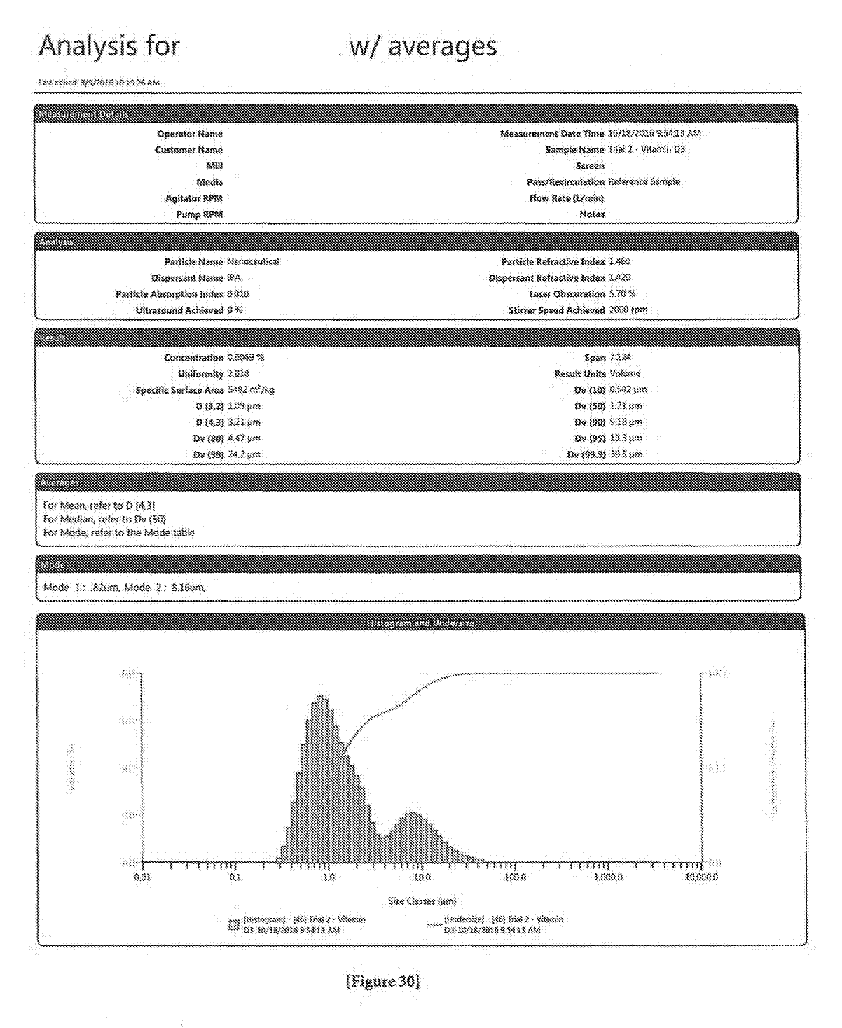

FIG. 31 is a graph showing experimental results for a batch size reduction process.

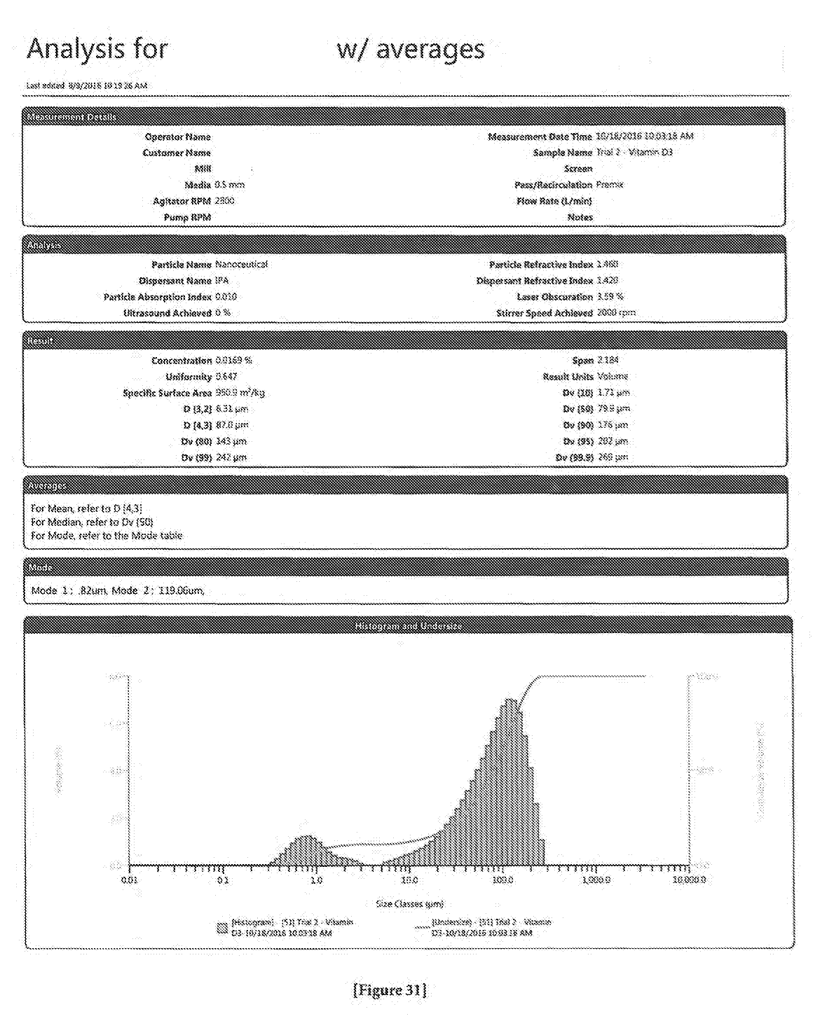

FIG. 32 is a graph showing experimental results for a batch size reduction process.

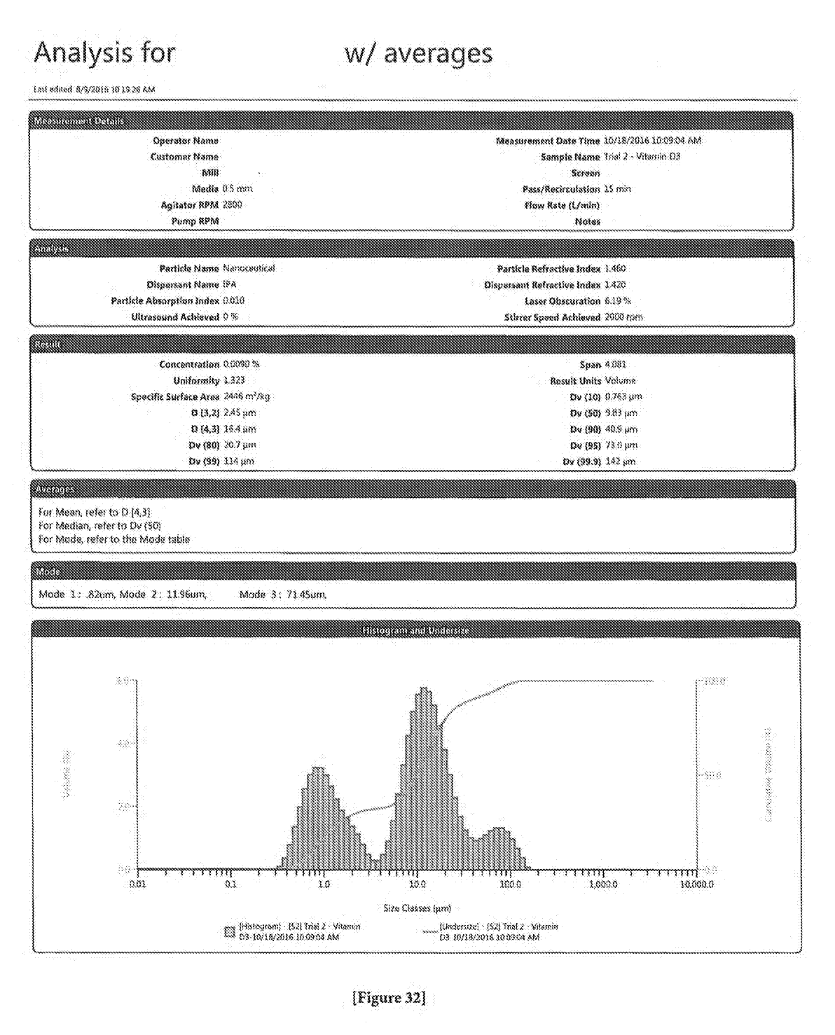

FIG. 33 is a graph showing experimental results for a batch size reduction process.

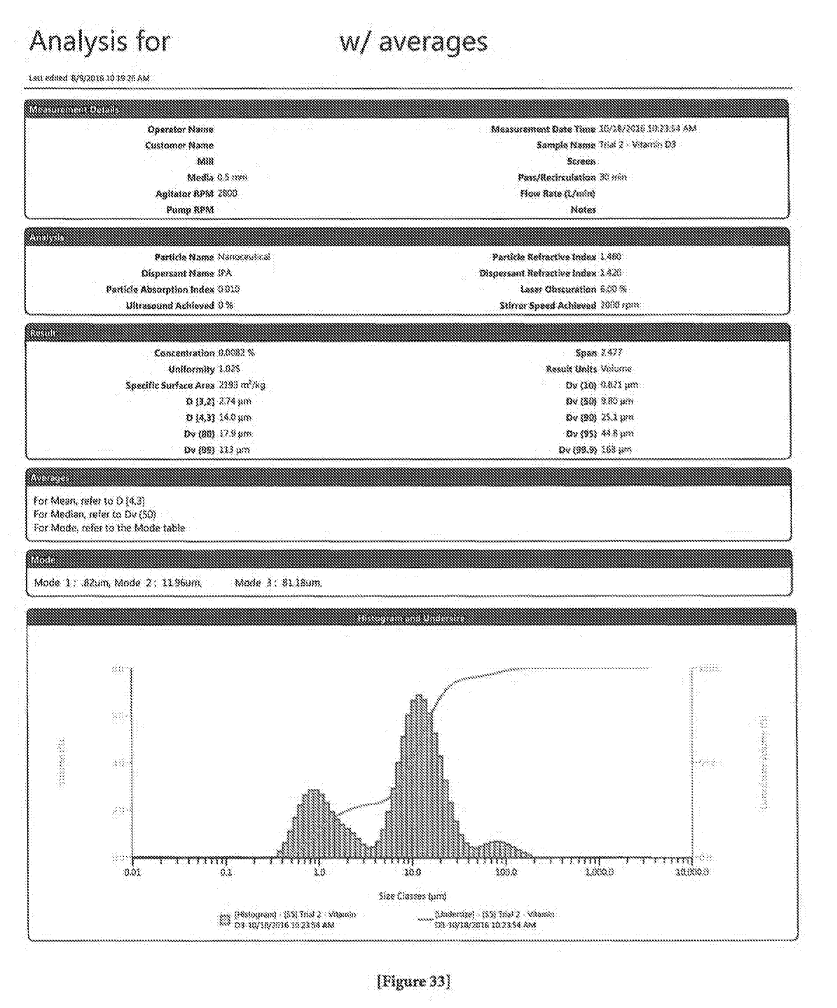

FIG. 34 is a graph showing experimental results for a batch size reduction process.

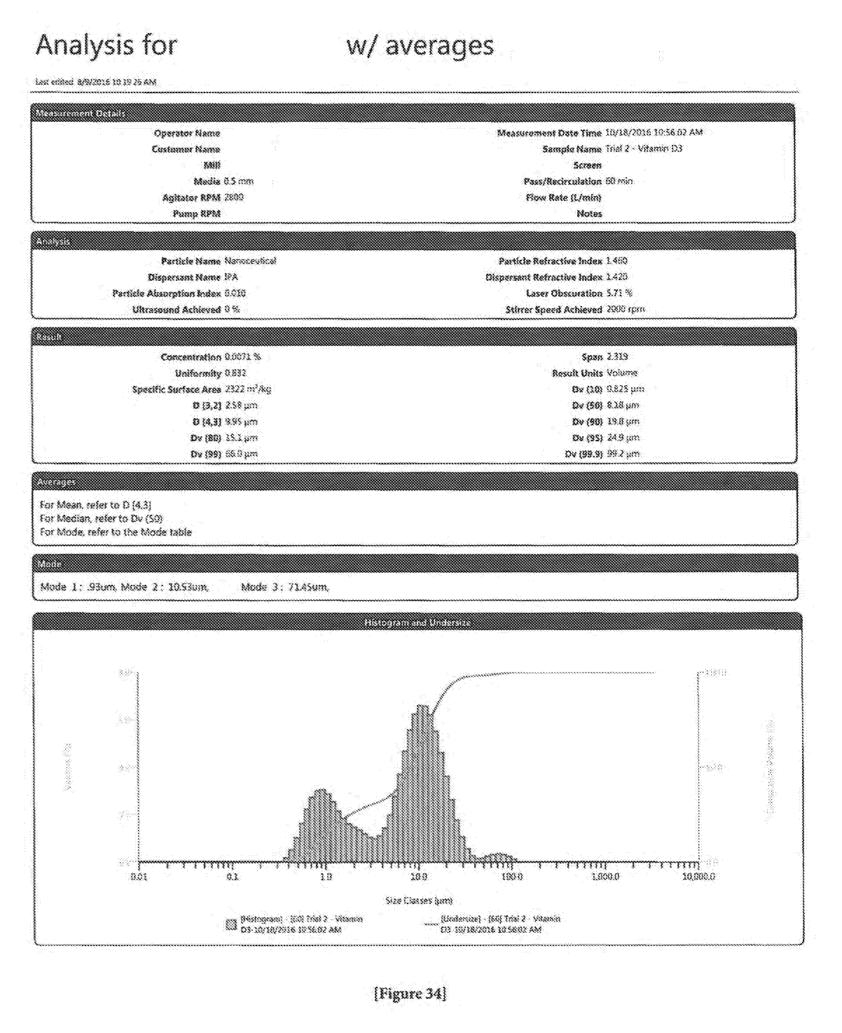

FIG. 35 is a graph showing experimental results for a batch size reduction process.

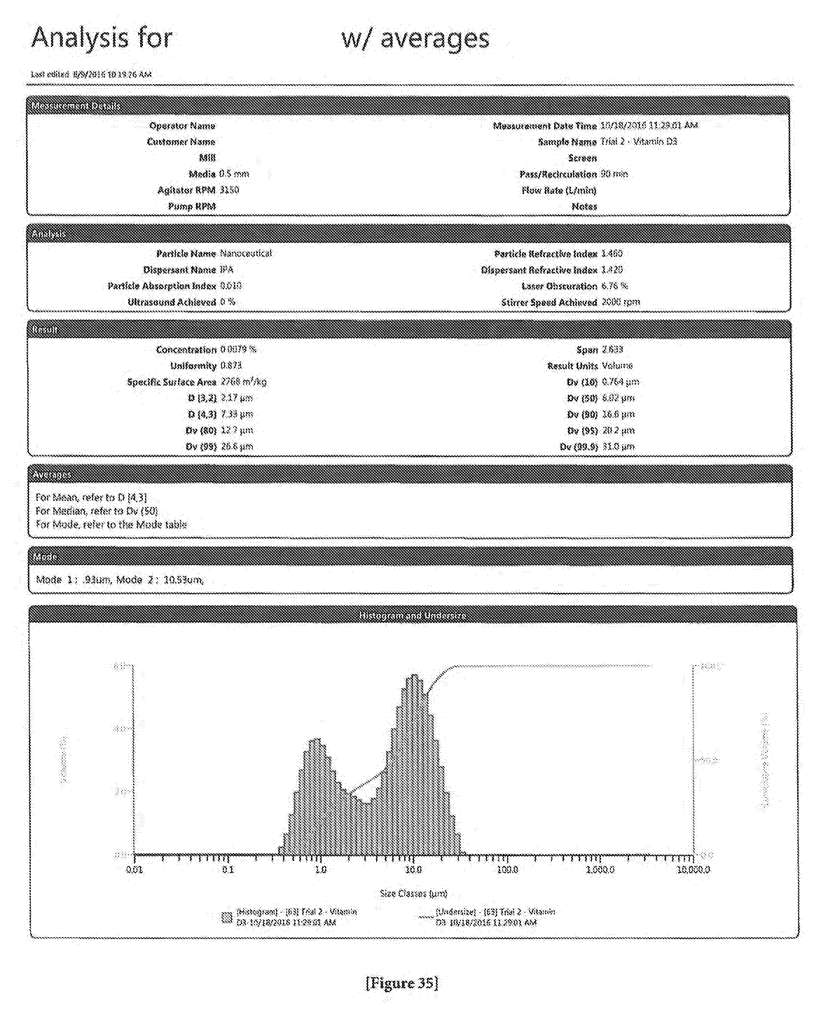

FIG. 36 is a graph showing experimental results for a batch size reduction process.

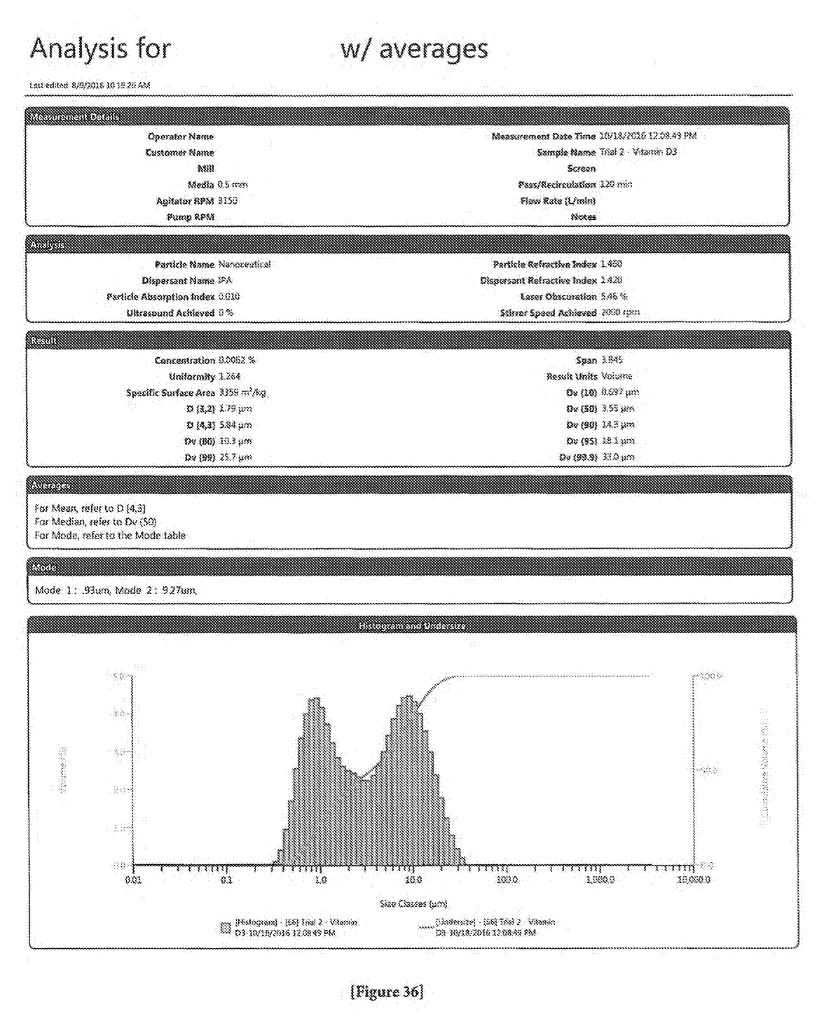

FIG. 37 is a graph showing experimental results for a batch size reduction process.

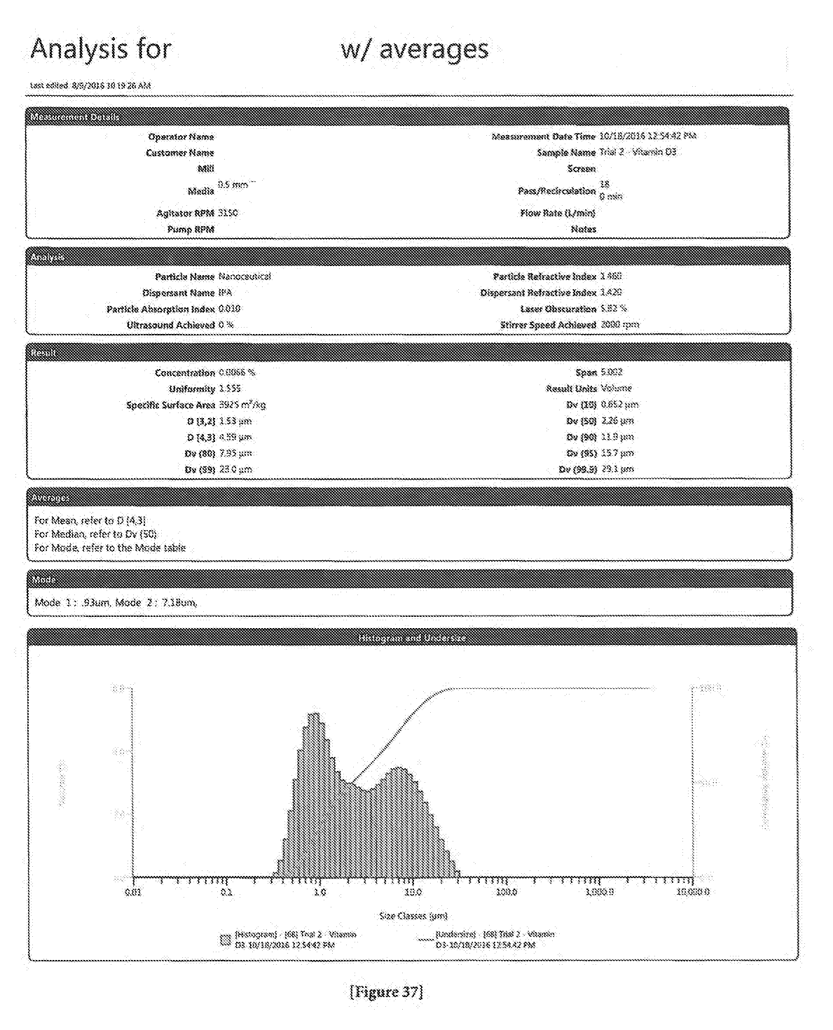

### DETAILED DESCRIPTION OF THE INVENTION

For reasons discussed above, there is a need to place glutathione in a state where it is stable and bioavailable. However, due to its tendency to oxidize and to agglomerate, it is difficult to handle in a stable and available manner. Further, there are no natural carriers of glutathione into human cells; rather, cells produce what glutathione they need. However, to overcome illness or amplify glutathione's beneficial effects, supplementing glutathione in certain circumstances is desired.

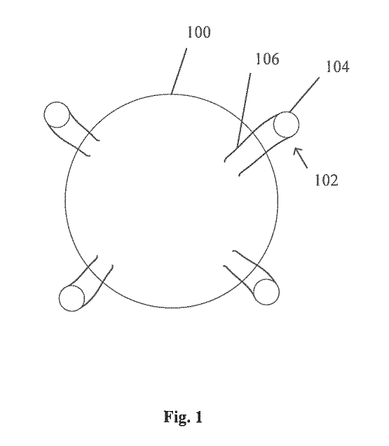

It has been discovered that particles of glutathione in the micron range are capable of penetrating cell walls. This penetration is aided by proper selection of chelating and surfactant agents to assist their cellular uptake. FIG. 1 shows a particle of glutathione 100 engaged with lecithin 102 where the hydrophobic end 104 of the lecithin 102 engages the glutathione and the hydrophilic end 106 extends out therefrom facilitating the particles uptake in aqueous environments.

Experiments on permeation of glutathione prepared according to the method through sheep mucosal membranes were performed, and they show excellent ability of the glutathione. The experiments were conducted by using vertical type diffusion cell (Franz type) having receptor compartment 60 ml volume with 10.18 cm² area. The receptor compartment was filled 60 ml of phosphate buffer pH 7.4; the activated dialysis membrane was mounted on the flange of the diffusion cell receptor compartment. The prepared Transdermal patch with surface area 10.18 cm² placed on center of membrane, the donor compartment was then placed in position and the two valves of the cell clamped together. The whole assembly was kept on a magnetic stirrer and solution in the receptor compartment was constantly and continuously stirred using a magnetic bead and at 37±1° C. maintained.

The permeation study of gel through the Sheep Mucosal Membrane was performed using Franz diffusion cell and membrane assembly, at 37° C.±1° C. and 50 rpm. This temperature and rpm was maintained by magnetic stirrer. The tissue was stored in Krebs buffer at 4° C. upon collection. After the membrane was equilibrated for 30 min with the buffer solution between both the chambers, the receiver chamber was filled with fresh buffer solution (pH 7.4), and the donor chamber was charged with 5 mL (mg/mL) of drug solution. Aliquots (5 mL) were collected at predetermined time intervals up to 45 min and the amount of drug permeated through the mucosa was then determined by measuring the values at 215 nm using HPLC method. The medium of the same volume (5 mL), which was pre-warmed at 37° C., was then replaced into the receiver chamber.

The results of three separate experiments conducted in the above-noted manner are summarized in the table, below. Within 30 seconds, approximately 50% of the glutathione had penetrated the membrane, and penetration increased to approximately 60% over 45 minutes (2700 seconds).

| | % drug | % drug | % drug |
|---|---|---|---|
| Time(sec) | diffused F1 | diffused F2 | diffused F3 |
| 30 | 51.63053593 | 48.53871429 | 48.22732044 |
| 60 | 54.10241137 | 51.12764804 | 49.66482929 |
| 90 | 58.75254979 | 52.48998882 | 50.53260164 |
| 120 | 59.90379893 | 52.71250507 | 51.59755103 |
| 150 | 61.31640799 | 53.25095571 | 53.51808652 |
| 180 | 62.02646213 | 53.65916581 | 55.98630023 |
| 300 | 62.03141279 | 53.79775511 | 56.25533515 |
| 600 | 62.45213137 | 54.00325162 | 57.04656261 |
| 1200 | 62.53731795 | 55.91558483 | 57.43133761 |
| 1800 | 62.79817987 | 56.01298695 | 59.60204276 |
| 2700 | 65.46283828 | 57.24034157 | 63.14885681 |

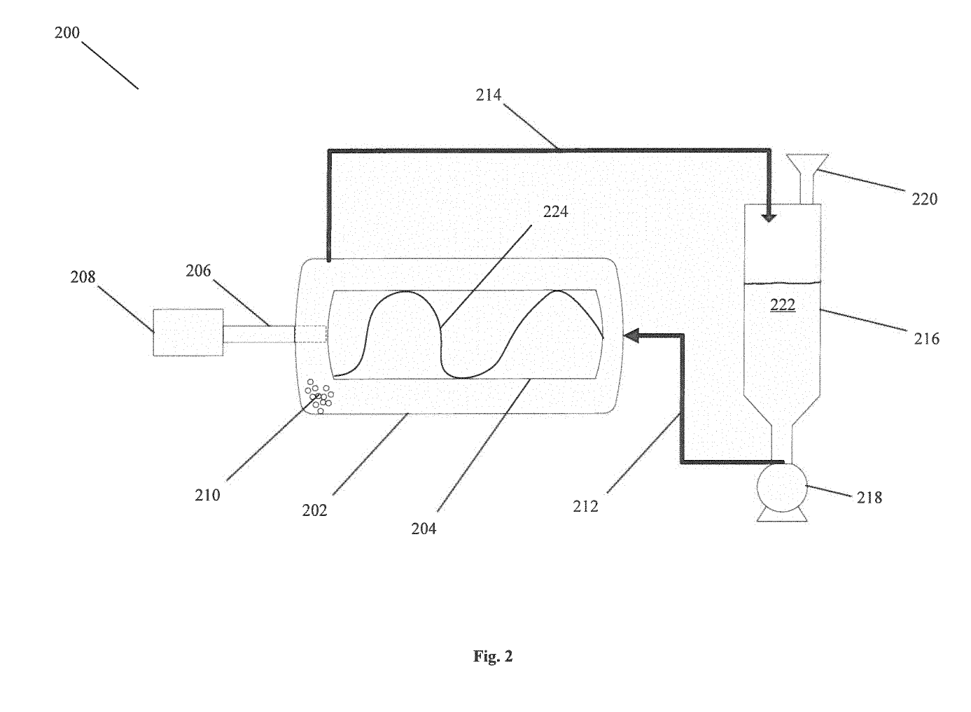

FIG. 2 shows one processing vessel, in this case, a batch process, that may be used for reducing the average size of glutathione particles and for admixing of at least one de-agglomeration agent. A grinding mill system 200 includes a chamber 202 having an agitator 204 therein. The agitator is powered by motor 208 via shaft 206. Contained within the vessel are beads 110 for grinding solids passing through the vessel. The beads are preferably zirconium dioxide (ZrO₂).

A tank 216 is charged with a mixture 212 of a fluid and a solid material to be processed, which is primarily glutathione in the present instance. In the step of immersion, an edible oil selected from, for example, almond, coconut, grapeseed, olive, and vegetable is added to the tank 216. Glutathione to be processed is added to the tank 216 via a funnel 220. The mixture 222 is forced into the chamber 202 through the inlet 212 by pump 218.

Upon entering the chamber 202, the mixture 222 encounters beads 210 in a bed agitated and fluidized by the agitator 204. To enhance agitation, an agitator profile 224 is provided on an outer surface of the agitator 204, which preferably extends along an entire length of the chamber 202. Substantially the entire volume of the chamber 202 is filled with beads 210. The beads engage the solid material between adjacent beads causing size reduction by the accompanying physical compression and grinding of the solid material.

In the step where at least one de-agglomeration agent is added to the mixture, the agents are also added via the funnel 220. Preferably, the de-agglomeration agent is added before the desired particle size reduction is complete. When added before the process is completed, the de-agglomeration agent is more thoroughly dispersed and placed in physical contact with the glutathione to engage it in the desired fashion, having the additional desired benefit of reducing processing time by preventing re-agglomeration of particles that have been reduced in size by engagement with beads.

Size reduction is specifically shown via a bed of grinding beds, and this method is preferred, but there are several technologies for producing fine particles such as a diamond chamber and microfluidizer. The common element in these systems is generally high shear and friction used to reduce particle size. When beads are used, zirconium oxide beads may be used, but other materials such as aluminum oxide ceramics may be appropriate. Bead sizes may be from 0.1-2 mm, but the range of 0.4-1.3 mm is preferred. A filter prevents the beads from leaving the chamber 202.

The glutathione is preferably immersed in an edible oil. Application of glutathione may be to the skin (transdermal), in which case these oils have lower tendency to irritate the skin. Transdermal application may occur via a cream rubbed onto the skin or via a transdermal patch, which is an adhesive patch placed on the skin to deliver a specific dose of medication through the skin and into the bloodstream. Alternatively, application may be sublingual or oral, in which case they must be edible to safely administered. Application rectally also generally requires a substance that is safe to ingest. Other methods in the same vein include inhaled and vaginal application. The oils may include the at least one of almond, coconut, grapeseed, olive, and vegetable.

Treatment of the glutathione with lecithin in oil with particle size reduction results in creation of a micelle-like or liposome-like structure upon introduction of the mixture (oil, glutathione and lecithin) into the aqueous digestive tract. The nature of the micelle or liposome-like structures facilitates its absorption through the digestive tract rather than the glutathione being digested in the alimentary canal.

Reducing the average size of glutathione particles to not more than 50 microns, and preferably 1 micron or less, is facilitated by adding a de-agglomeration agent. When the de-agglomeration agent is lecithin, the additional advantages noted above regarding the ability to apply glutathione transdermally, sublingually, orally, rectally, by inhaler, and even vaginally, and the like are provided. The prior art teaches that oral administration is impractical, but the present invention overcomes the impediments that previously taught against such uses.

The de-agglomeration agent may comprise at least one selected from surfactants or chelating agents. Lecithin has been discussed as a preferred surfactant. Lecithin acts as an amphipathic surfactant, and other surfactants of this type could also be substituted for lecithin.

Chelating agents assist in cell permeation and may be used in addition to or instead of a surfactant. Preferably, if a chelating agent is used, it is used in addition to a surfactant. Chelating agents that may be used include ethylenediaminetetraacetic acid (EDTA). However, due to the potential dangers of EDTA and other chelating agents to strip calcium from the body, care must be taken to ensure that treatment does not present the danger of hypocalcemia or the like.

Having thus described the invention, I claim:

1. A method of economically producing stable and bio-available glutathione comprising:
a. immersing glutathione particles in an edible oil;
b. reducing the average size of glutathione particles to not more than 50 microns; and
c. adding a de-agglomeration agent.

2. The method of claim 1 further comprising:
a. the oil selected from at least one of almond, coconut, grapeseed, olive, and vegetable;
b. for the reducing step using a process selected from at least one of diamond chamber, grinding beads, and microfluidizer; and
c. the de-agglomeration agent comprising at least one selected from surfactants or chelating agents.

3. The method of claim 2 further comprising:
a. the reducing step comprising grinding beads have a media size from 0.4-1.3 mm to reduce the average size of glutathione particles to not more than 1 micron; and
b. the de-agglomeration agent comprising lecithin.

4. The method of claim 1 further comprising adding the de-agglomeration agent before the particle size reduction is complete, whereby the de-agglomeration agent serves to speed the reduction of size reduction by preventing agglomeration of particles during processing.

5. The method of claim 4 further comprising:
a. the oil selected from at least one of almond, coconut, grapeseed, olive, and vegetable;
b. for the reducing step, processing in a batch manner using a process selected from at least one of diamond chamber and grinding beads; and
c. the de-agglomeration agent comprising at least one selected from surfactants or chelating agents.

6. The method of claim 5 further comprising:
a. the reducing step using grinding beads have a media size from 0.4-1.3 mm to reduce the average size of glutathione particles to not more than 1 micron; and
b. the de-agglomeration agent comprising lecithin.

7. The method of claim 6 further comprising adding the lecithin during the reducing step in more than one batch at different stages during the size-reduction step.

8. A method of administering bio-available glutathione to a patient comprising:
a. immersing glutathione particles in an edible oil;
b. reducing the average size of glutathione particles to not more than 50 microns;
c. adding a de-agglomeration agent;
d. administering a desired dose to the patient by a method selected from oral, pulmonary, rectal, sublingual, transdermal, and vaginal.

9. The method of claim 8 further comprising:
a. the oil selected from at least one of almond, coconut, grapeseed, olive, and vegetable;
b. for the reducing step using a process selected from at least one of diamond chamber, grinding beads, and microfluidizer; and
c. the de-agglomeration agent comprising at least one selected from surfactants or chelating agents.

10. The method of 9 further comprising transdermal application via a transdermal patch.
